(19) United States
(12) Patent Application Publication
(10) Pub. No.: US 2024/0081743 A1
(43) Pub. Date: Mar. 14, 2024

(54) SENSOR IMPLANT DEVICE ANCHORING

(71) Applicant: EDWARDS LIFESCIENCES CORPORATION, Irvine, CA (US)

(72) Inventors: Scott Louis Pool, Laguna Hills, CA (US); Arvin T. Chang, Yorba Linda, CA (US)

(21) Appl. No.: 18/513,199

(22) Filed: Nov. 17, 2023

Related U.S. Application Data
(63) Continuation of application No. PCT/US2022/030211, filed on May 20, 2022.
(60) Provisional application No. 63/225,039, filed on Jul. 23, 2021.
(60) Provisional application No. 63/224,286, filed on Jul. 21, 2021.
(60) Provisional application No. 63/191,534, filed on May 21, 2021.
(60) Provisional application No. 63/235,038, filed on Aug. 19, 2021.

(51) Int. Cl. A61B 5/00; A61B 90/00

(52) U.S. Cl. CPC A61B 5/6882; A61B 5/6869; A61B 5/6876; A61B 90/08; A61B 2090/0815; A61B 2560/0462; A61B 2562/16

(57) ABSTRACT

A sensor implant device comprises a sensor body, a sensor component, and one or more anchoring features coupled to the sensor body and configured to anchor within a blood flow pathway or left atrial appendage.

### RELATED APPLICATION

This application is a continuation of International Patent Application No. PCT/US2022/030211, filed May 20, 2022, and entitled SENSOR IMPLANT DEVICE ANCHORING, which claims priority to U.S. Provisional Patent Application Ser. No. 63/191,534, filed on May 21, 2021 and entitled IMPLANT-COUPLED SENSORS, U.S. Provisional Patent Application Ser. No. 63/224,286, filed on Jul. 21, 2021 and entitled IMPLANT-ADJACENT SENSOR ANCHORING, U.S. Provisional Patent Application Ser. No. 63/225,039, filed on Jul. 23, 2021 and entitled SHUNT BARREL SENSOR IMPLANT ANCHORING, and U.S. Provisional Patent Application Ser. No. 63/235,038, filed on Aug. 19, 2021 and entitled SENSOR IMPLANT DEVICE ANCHORING, the complete disclosures of which are hereby incorporated by reference in their entirety.

### BACKGROUND

### Field

The present disclosure generally relates to the field of a medical implant devices.

### Description of Related Art

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with certain chambers and/or vessels of the heart, such as the left atrium, using one or more sensor implant devices.

For purposes of summarizing the disclosure, certain aspects, advantages, and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIG. 2 illustrates example pressure waveforms associated with various chambers and vessels of the heart according to one or more examples.

FIG. 3 illustrates a graph showing left atrial pressure ranges.

FIG. 4 is a block diagram representing an implant device in accordance with one or more examples.

FIG. 5 is a block diagram representing a system for monitoring one or more physiological parameters associated with a patient according to one or more examples.

FIG. 6 illustrates an example sensor assembly/device that can be a component of a sensor implant device, in accordance with one or more examples.

FIG. 7 provides an overhead view of a sensor implant device in accordance with one or more examples.

FIG. 8 provides a side view of a sensor implant device in accordance with one or more examples.

FIG. 9 provides a side view of a sensor implant device in accordance with one or more examples.

FIG. 10 provides a side view of a sensor implant device implanted and/or anchored within a heart in accordance with one or more examples.

FIG. 11 provides a side view of another sensor implant device implanted and/or anchored within a heart in accordance with one or more examples.

FIG. 12 provides a side view of another sensor implant device implanted and/or anchored within a heart in accordance with one or more examples.

FIG. 13 illustrates a sensor implant device comprising a stent-like anchoring feature in accordance with one or more examples.

FIG. 14 illustrates a sensor implant device comprising a stent-like anchoring feature implanted and/or anchored within a heart in accordance with one or more examples.

FIG. 15 illustrates another sensor implant device comprising a stent-like anchoring feature implanted and/or anchored within a heart in accordance with one or more examples.

FIG. 16 illustrates a sensor implant device comprising a clip anchoring feature in accordance with one or more examples.

FIG. 17 illustrates a sensor implant device comprising a clip anchoring feature implanted and/or anchored within a left atrial appendage in accordance with one or more examples.

FIG. 18 illustrates a sensor implant device comprising a coil anchoring feature in accordance with one or more examples.

FIG. 19 illustrates a sensor implant device comprising a coil anchoring feature implanted and/or anchored within a left atrial appendage in accordance with one or more examples.

FIG. 20A illustrates a sensor implant device comprising one or more hook anchoring features in a collapsed state in accordance with one or more examples.

FIG. 20B illustrates a sensor implant device comprising one or more hook anchoring features in an expanded state in accordance with one or more examples.

FIG. 20C provides an overhead view of a sensor implant device comprising one or more hook anchoring features in an expanded state in accordance with one or more examples.

FIG. 20D illustrates a sensor implant device comprising one or more hook anchoring features in an expanded state implanted and/or anchored in a left atrial appendage in accordance with one or more examples.

FIG. 21 illustrates a sensor implant device comprising a coil anchoring feature implanted and/or anchored within a blood flow pathway in accordance with one or more examples.

FIG. 22 illustrates another sensor implant device comprising a coil anchoring feature implanted and/or anchored within a blood flow pathway in accordance with one or more examples.

FIG. 23 illustrates another sensor implant device comprising a coil anchoring feature implanted and/or anchored within a blood flow pathway in accordance with one or more examples.

FIG. 24 illustrates a sensor implant device comprising a coil anchoring feature and a covering implanted and/or anchored within a blood flow pathway in accordance with one or more examples.

FIG. 25 illustrates a sensor implant device comprising a coil anchoring feature implanted and/or anchored within a left atrial appendage in accordance with one or more examples.

FIG. 26 illustrates a sensor implant device comprising a coil anchoring feature implanted and/or anchored within a blood flow pathway in accordance with one or more examples.

FIG. 27A and FIG. 27B illustrate another sensor implant device comprising a coil anchoring feature implanted and/or anchored within a blood flow pathway in accordance with one or more examples.

FIG. 28 illustrates a sensor implant device and associated delivery systems in accordance with one or more examples.

FIG. 29 (FIGS. 29-1, 29-2, 29-3, and 29-4) provides a flowchart illustrating a process including one or more steps for delivering one or more implants and/or sensors, in accordance with one or more examples.

FIG. 30 (FIGS. 30-1, 30-2, 30-3, and 30-4) provides images corresponding to steps of the process of FIG. 29.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “cupper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for monitoring of one or more physiological parameters of a patient (e.g., blood pressure) using sensor-integrated cardiac shunts and/or other medical implant devices. In some implementations, the present disclosure relates to cardiac shunts and/or other cardiac implant devices that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain examples are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy.

While the various sensor devices described herein may be integrated with the various medical implant devices described herein, the sensor devices may be separate devices from the medical implant devices. For example, a sensor device may form a breakable and/or releasable connection with a medical implant device. Moreover, sensor devices described herein may be configured to be delivered separately (e.g., before and/or after) medical implant devices within a heart of a patient. For example, a sensor device may not be attached to a medical implant device during delivery processes (e.g., during delivery through a catheter) of the sensor device and/or medical implant device but may be attached/coupled to the medical implant device following delivery (e.g., following removal from a catheter) to a desired location within the heart. Example delivery locations can include the left atrium, the left atrial appendage, the pulmonary vein, the coronary sinus, and/or the various tissue walls associated with these locations.

In some examples, a catheter and/or guidewire used for delivering a sensor device may also be used for delivering a medical implant device. For example, the catheter and/or guidewire may remain within the body following delivery of the sensor device and/or medical implant device for delivery of the remaining device(s).

Some sensor devices described herein may be configured to be delivered prior to delivery of the medical implant devices described herein. This may advantageously simplify delivery of the sensor devices and/or medical implant devices and/or may provide for simple imaging the sensor devices and/or medical implant devices. The sensor devices may be adjusted as necessary to maximize measurements of the sensor devices prior to delivery of the medical implant devices. Moreover, delivery of the medical implant devices may be delayed and/or aborted as needed following delivery of the sensor devices.

Some sensor devices described herein may be configured to be delivered after delivery of the medical implant devices described herein. This may advantageously simplify delivery of the sensor devices and/or medical implant devices and/or may provide for simple imaging the sensor devices and/or medical implant devices. The sensor devices may be adjusted as necessary to maximize measurements of the sensor devices. Moreover, the sensor device may be effectively secured to the medical implant devices with minimal risk of dislodgement of the sensor devices.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

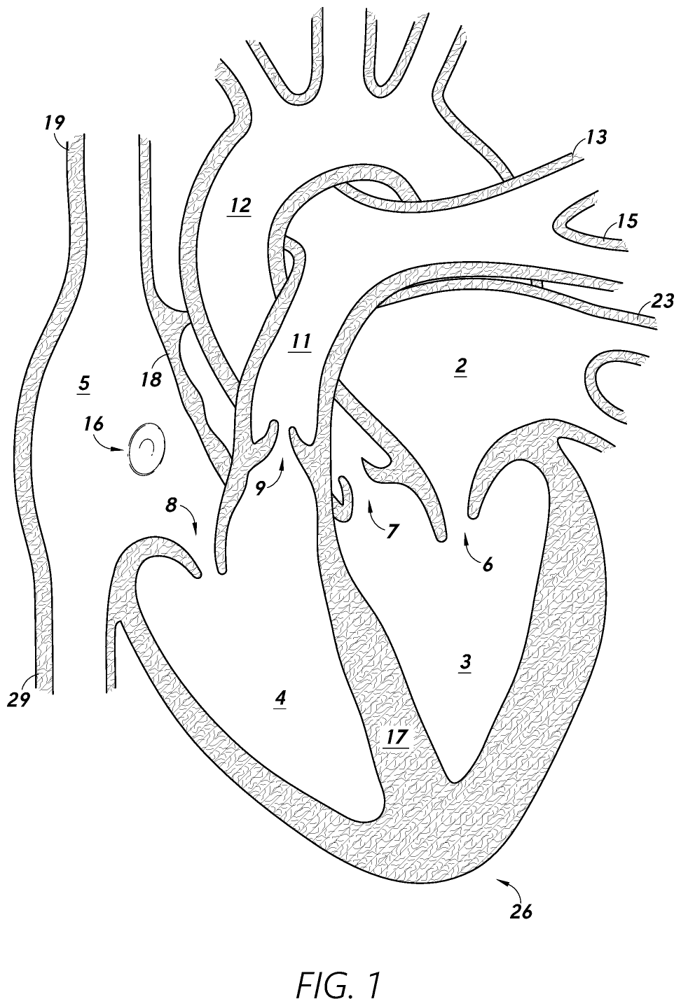

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 14 into the heart 1. Other access routes may also be used, and each can utilize a percutaneous incision through which the guidewire and catheter are inserted into the vasculature, normally through a sealed introducer, and from there the physician can control the distal ends of the devices from outside the body.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy. As described above, pressure buildup in one or more chambers or areas of the heart can be associated with congestive heart failure. Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

The present disclosure provides systems, devices, and methods for guiding the administration of medication relating to the treatment of congestive heart failure at least in part by directly monitoring pressure in the left atrium, or other chamber or vessel for which pressure measurements are indicative of left atrial pressure and/or pressure levels in one or more other vessels/chambers, such as for congestive heart failure patients in order to reduce hospital readmissions, morbidity, and/or otherwise improve the health prospects of the patient.

### Cardiac Pressure Monitoring

Cardiac pressure monitoring in accordance with examples of the present disclosure may provide a proactive intervention mechanism for preventing or treating congestive heart failure and/or other physiological conditions. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with examples of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to examples of the present disclosure, normal ventricular filling pressures may advantageously be maintained, thereby preventing or reducing effects of heart failure, such as dyspnea.

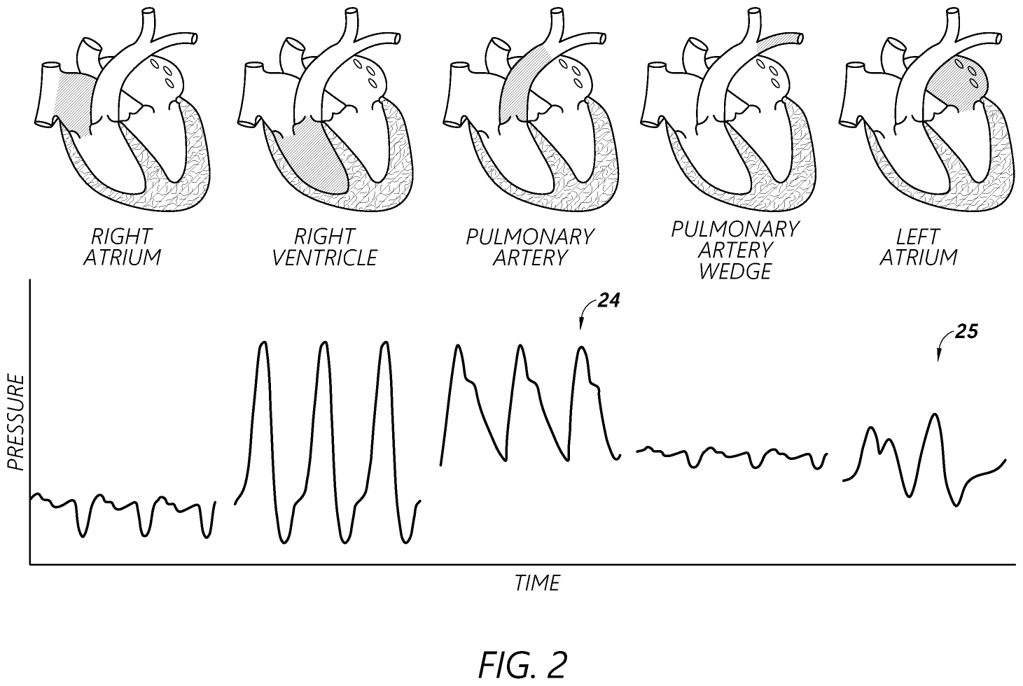

As referenced above, with respect to cardiac pressures, pressure elevation in the left atrium may be particularly correlated with heart failure. FIG. 2 illustrates example pressure waveforms associated with various chambers and vessels of the heart according to one or more examples. The various waveforms illustrated in FIG. 2 may represent waveforms obtained using right heart catheterization to advance one or more pressure sensors to the respective illustrated and labeled chambers or vessels of the heart. As illustrated in FIG. 2, the waveform 25, which represents left atrial pressure, may be considered to provide the best feedback for early detection of congestive heart failure. Furthermore, there may generally be a relatively strong correlation between increases and left atrial pressure and pulmonary congestion.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 24, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to the other pressure waveforms shown in FIG. 2. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with examples of the present disclosure may be used to predict heart failure up two weeks or more before the manifestation of symptoms or markers of heart failure (e.g., dyspnea). When heart failure predictors are recognized using cardiac pressure sensor examples in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

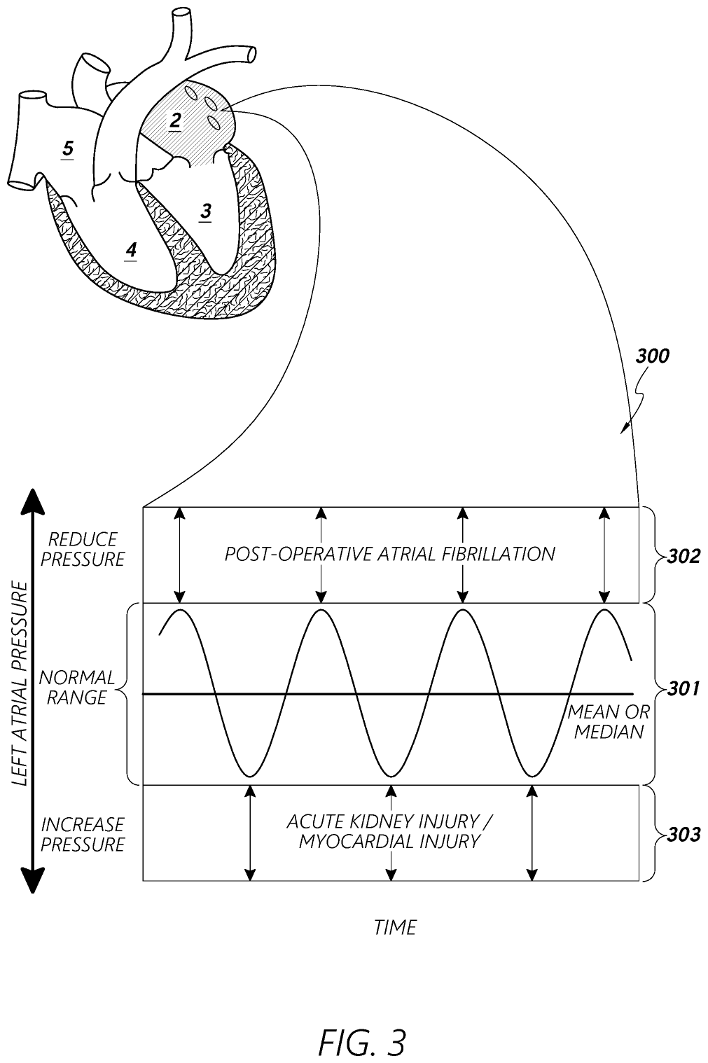

FIG. 3 illustrates a graph 300 showing left atrial pressure ranges including a normal range 301 of left atrial pressure that is not generally associated with substantial risk of postoperative atrial fibrillation, acute kidney injury, myocardial injury, heart failure and/or other health conditions. Examples of the present disclosure provide systems, devices, and methods for determining whether a patient's left atrial pressure is within the normal range 301, above the normal range 303, or below the normal range 302 through the use of certain sensor implant devices. For detected left atrial pressure above the normal range, which may be correlated with an increased risk of heart failure, examples of the present disclosure as described in detail below can inform efforts to reduce the left atrial pressure until it is brought within the normal range 301. Furthermore, for detected left atrial pressure that is below the normal range 301, which may be correlated with increased risks of acute kidney injury, myocardial injury, and/or other health complications, examples of the present disclosure as described in detail below can serve to facilitate efforts to increase the left atrial pressure to bring the pressure level within the normal range 301.

Implant Devices with Integrated Sensors

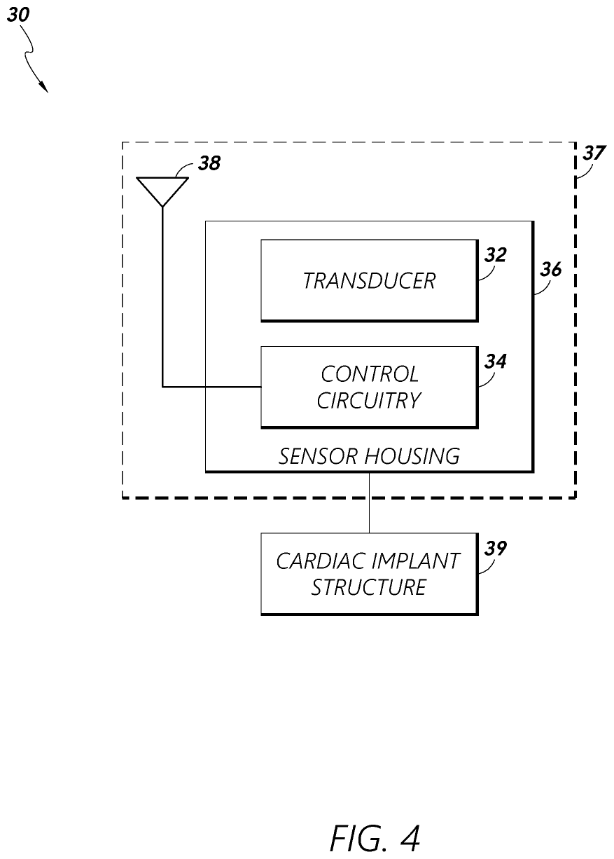

In some implementations, the present disclosure relates to sensors associated or integrated with cardiac shunts or other implant devices. Such integrated devices may be used to provide controlled and/or more effective therapies for treating and preventing heart failure and/or other health complications related to cardiac function. FIG. 4 is a block diagram illustrating an implant device 30 comprising a shunt (or other type of implant) structure 39. In some examples, the shunt structure 39 is physically integrated with and/or connected to a sensor device 37. The sensor device 37 may be, for example, a pressure sensor, or other type of sensor. In some examples, the sensor 37 comprises a transducer 32, such as a pressure transducer, as well as certain control circuitry 34, which may be embodied in, for example, an application-specific integrated circuit (ASIC).

The control circuitry 34 may be configured to process signals received from the transducer 32 and/or communicate signals associated therewith wirelessly through biological tissue using the antenna 38. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry. The transducer(s) 32 and/or antenna(s) 38 can be considered part of the control circuitry 34.

The antenna 38 may comprise one or more coils or loops of conductive material, such as copper wire or the like. In some examples, at least a portion of the transducer 32, control circuitry 34, and/or the antenna 38 are at least partially disposed or contained within a sensor housing 36, which may comprise any type of material, and may advantageously be at least partially hermetically sealed. For example, the housing 36 may comprise glass or other rigid material in some examples, which may provide mechanical stability and/or protection for the components housed therein. In some examples, the housing 36 is at least partially flexible. For example, the housing may comprise polymer or other flexible structure/material, which may advantageously allow for folding, bending, or collapsing of the sensor 37 to allow for transportation thereof through a catheter or other introducing means.

The transducer 32 may comprise any type of sensor means or mechanism. For example, the transducer 32 may be a force-collector-type pressure sensor. In some examples, the transducer 32 comprises a diaphragm, piston, bourdon tube, bellows, or other strain- or deflection-measuring component(s) to measure strain or deflection applied over an area/surface thereof. The transducer 32 may be associated with the housing 36, such that at least a portion thereof is contained within or attached to the housing 36. With respect to sensor devices/components being “associated with” a stent or other implant structure, such terminology may refer to a sensor device or component being physically coupled, attached, or connected to, or integrated with, the implant structure.

In some examples, the transducer 32 comprises or is a component of a piezoresistive strain gauge, which may be configured to use a bonded or formed strain gauge to detect strain due to applied pressure, wherein resistance increases as pressure deforms the component/material. The transducer 32 may incorporate any type of material, including but not limited to silicon (e.g., monocrystalline), polysilicon thin film, bonded metal foil, thick film, silicon-on-sapphire, sputtered thin film, and/or the like.

In some examples, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some examples, the transducer 32 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz.

In some examples, the transducer 32 comprises or is a component of a strain gauge. For example, a strain gauge example may comprise a pressure sensitive element on or associated with an exposed surface of the transducer 32. In some examples, a metal strain gauge is adhered to a surface of the sensor, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

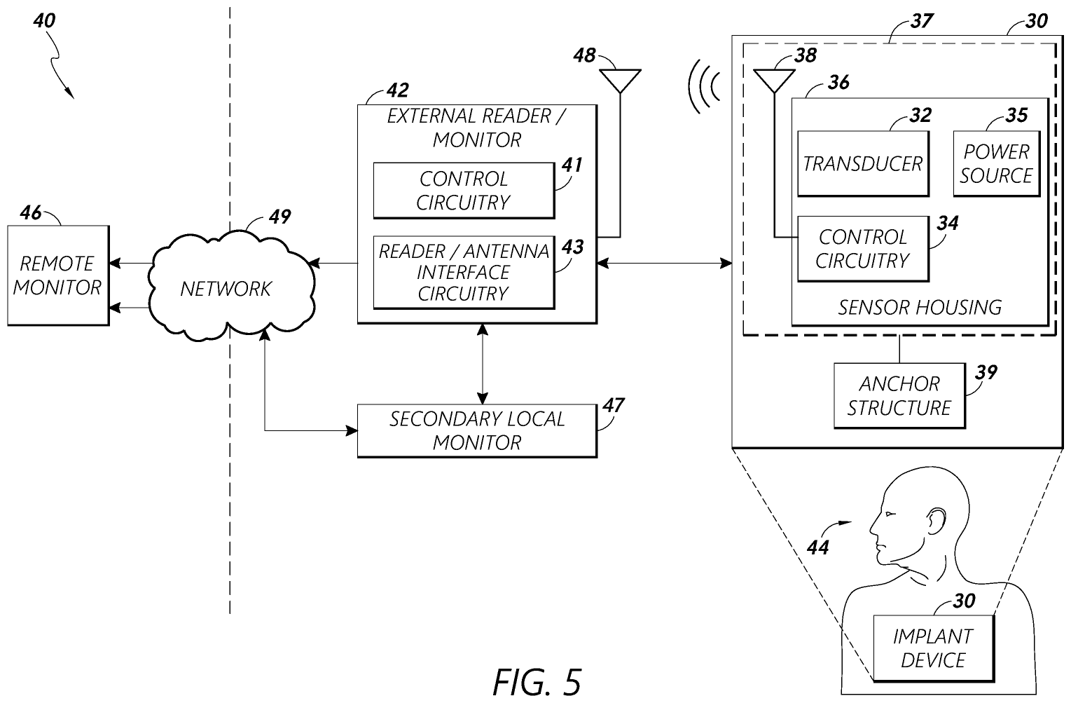

FIG. 5 shows a system 40 for monitoring one or more physiological parameters (e.g., left atrial pressure and/or volume) in a patient 44 according to one or more examples. The patient 44 can have a medical implant device 30 implanted in, for example, the heart (not shown), or associated physiology, of the patient 44. For example, the implant device 30 can be implanted at least partially within the left atrium and/or coronary sinus of the patient's heart. The implant device 30 can include one or more sensor transducers 32, such as one or more microelectromechanical system (MEMS) devices (e.g., MEMS pressure sensors, or other type of sensor transducer).

In certain examples, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry 41. In certain examples, both the internal 30 and external 42 subsystems include a corresponding coil antenna for wireless communication and/or power delivery through patient tissue disposed therebetween. The sensor implant device 30 can be any type of implant device. For example, in some examples, the implant device 30 comprises a pressure sensor integrated with another functional implant structure 39, such as a prosthetic shunt or stent device/structure.

Certain details of the implant device 30 are illustrated in the enlarged block 30 shown. The implant device 30 can comprise an implant/anchor structure 39 as described herein. For example, the implant/anchor structure 39 can include a percutaneously-deliverable shunt device configured to be secured to and/or in a tissue wall to provide a flow path between two chambers and/or vessels of the heart, as described in detail throughout the present disclosure. Although certain components are illustrated in FIG. 5 as part of the implant device 30, it should be understood that the sensor implant device 30 may only comprise a subset of the illustrated components/modules and can comprise additional components/modules not illustrated. The implant device may represent an example of the implant device shown in FIG. 4, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some examples, the transducer 32 comprises a diaphragm, piston, bourdon tube, bellows, or other strain- or deflection-measuring component(s) to measure strain or deflection applied over an area/surface thereof. The transducer 32 may be associated with the sensor housing 36, such that at least a portion thereof is contained within, or attached to, the housing 36.

In some examples, the transducer 32 comprises or is a component of a strain gauge, which may be configured to use a bonded or formed strain gauge to detect strain due to applied pressure. For example, the transducer 32 may comprise or be a component of a piezoresistive strain gauge, wherein resistance increases as pressure deforms the component/material of the strain gauge. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some examples, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some examples, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some examples, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz.

In some examples, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain examples, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some examples.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions from the implant device 30 and/or provide wireless power to the implant device 30 using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas, or the like for treatment/processing of transmitted signals over the network 49 and/or for receiving signals from the implant device 30. In certain examples, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain examples, the local monitor 42 comprises a smartphone, laptop computer, or other mobile computing device, or any other type of computing device.

In certain examples, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or other external subsystem. In certain examples, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain examples, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain examples, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some examples, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain examples, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an example, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain examples disclosed herein describe communication with the remote monitor subsystem 46 from the implant device indirectly through the local monitor device 42, in certain examples, the implant device 30 can comprise a transmitter capable of communicating over the network 49 with the remote monitor subsystem 46 without the necessity of relaying information through the local monitor device 42.

In some examples, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some examples, which may provide mechanical stability and/or protection for the components housed therein. In some examples, the housing 36 is at least partially flexible. For example, the housing may comprise polymer or other flexible structure/material, which may advantageously allow for folding, bending, or collapsing of the sensor 37 to allow for transportation thereof through a catheter or other percutaneous introducing means.

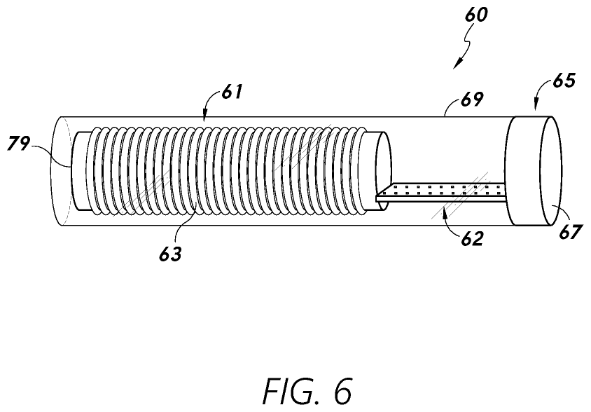

As referenced above, shunt and other implant devices/structures may be integrated with sensor, antenna/transceiver, and/or other components to facilitate in vivo monitoring of pressure and/or other physiological parameter(s). Sensor devices in accordance with examples of the present disclosure may be integrated with cardiac shunt structures/devices or other implant devices using any suitable or desirable attachment or integration mechanism or configuration. FIG. 6 illustrates an example sensor assembly/device 60 that can be a component of a sensor implant device. The sensor device 60 may be configured to provide sensor readings relating to one or more physiological parameters associated with a target implantation site.

The sensor device 60 may be configured for attachment to implant devices. For example, a coil form including one or more wires or other material or structure shaped into one or more winds of coil forming a fluid conduit/barrel portion and axial end flanges may be used to attach the sensor device 60 to one or more implants. A shunt structure may be integrated with pressure sensor functionality in accordance with certain examples disclosed herein. The shunt structure may be configured to hold the sensor device 60.

The sensor device 60 may advantageously be disposed, positioned, secured, oriented, and/or otherwise situated in a configuration in which a sensor transducer component 65 thereof is disposed within a channel area of a shunt structure. The term “channel area” is used herein according to its broad and ordinary meaning and may refer to a three-dimensional space defined by a radial boundary of a fluid conduit and extending axially from the fluid conduit.

In some examples, the sensor assembly 61 includes a sensor component 65 and an antenna component 69. The sensor component 65 may comprise any type of sensor device as described in detail above. In some examples, the sensor 65 may be attached to or integrated with an arm member of a shunt structure.

The sensor 65 includes a sensor element 67, such as a pressure sensor transducer. As described herein, the sensor assembly 61 may be configured to implement wireless data and/or power transmission. The sensor assembly 61 may include an antenna component 69 for such purpose. The antenna 69 may be contained at least partially within an antenna housing 79, which may further have disposed therein certain control circuitry configured to facilitate wireless data and/or power communication functionality. In some examples, the antenna component 69 comprises one or more conductive coils 62, which may facilitate inductive powering and/or data transmission. In examples comprising conductive coil(s), such coil(s) may be wrapped/disposed at least partially around a magnetic (e.g., ferrite, iron) core 63.

The antenna component 69 may be attached to, integrated with, or otherwise associated with an arm/anchor feature of a shunt structure.

The sensor assembly 61 may advantageously be biocompatible. For example, the sensor 65 and antenna 69 may comprise biocompatible housings, such as a housing comprising glass or other biocompatible material. However, at least a portion of the sensor element 67, such as a diaphragm or other component, may be exposed to the external environment in some examples in order to allow for pressure readings, or other parameter sensing, to be implemented. With respect to the antenna housing 79, the housing 79 may comprise an at least partially rigid cylindrical or tube-like form, such as a glass cylinder form. In some examples, the sensor 65/67 component is approximately 3 mm or less in diameter. The antenna 69 may be approximately 20 mm or less in length.

The sensor assembly 61 may be configured to communicate with an external system when implanted in a heart or other area of a patient's body. For example, the antenna 69 may receive power wirelessly from the external system and/or communicate sensed data or waveforms to and/or from the external system. The sensor assembly 61 may be attached to, or integrated with, a shunt structure in any suitable or desirable way. For example, in some implementations, the sensor 65 and/or antenna 69 may be attached or integrated with the shunt structure using mechanical attachment means. In some examples, the sensor 65 and/or antenna 69 may be contained in a pouch or other receptacle that is attached to a shunt structure.

The sensor element 67 may comprise a pressure transducer. For example, the pressure transducer may be a microelectromechanical system (MEMS) transducer comprising a semiconductor diaphragm component. In some examples, the transducer may include an at least partially flexible or compressible diaphragm component, which may be made from silicone or other flexible material. The diaphragm component may be configured to be flexed or compressed in response to changes in environmental pressure.

### Sensor Implant Devices

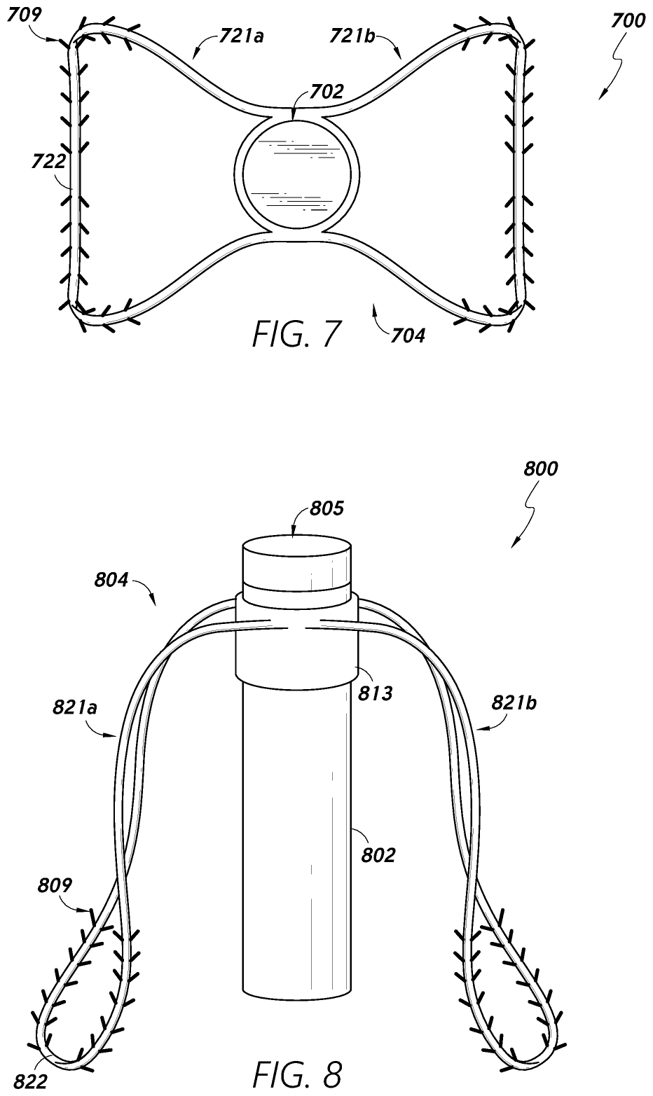

FIG. 7 provides an overhead view of a sensor implant device 700 in accordance with one or more examples. The sensor implant device 700 can comprise a sensor assembly/device 702 coupled, attached, and/or otherwise releasably and/or permanently secured to one or more anchoring features 704. The term “anchoring feature” is used herein in accordance with its plain and ordinary meaning and may refer to any means for anchoring, which can include one or more clips, puncture coils, hooks, arms, cords, and/or other features configured for anchoring and/or attachment at one or more areas of tissue within a heart.

The sensor device 702 may comprise at least one sensor component. The sensor component(s) may comprise any type of sensor device as described in detail above. The sensor implant device 700 may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 704 may be configured to extend in multiple directions around the sensor device 702 and/or may extend in generally opposite directions laterally (i.e., along a diameter of the sensor device 702) from the sensor device 702. The one or more anchoring features 704 may extend linearly and/or non-linearly away from and/or along the sensor device 702. In some examples, the one or more anchoring features 704 extend longitudinally along the sensor device 702 (e.g., along a shaft of the sensor device 702).

The one or more anchoring features 704 may comprise one or more lines, which can include wires and/or cords, forming one or more loops. The term “loop” is used herein in accordance with its plain and ordinary meaning and may refer to any length of material (e.g., a metallic and/or plastic cord) configured to extend from the sensor device 702 and re-connect to the sensor device 702 with or without gaps in the material. In some examples, the one or more loops may be configured to extend across one or more blood flow pathways of a heart and/or to contact one or more tissue walls. The one or more anchoring features 704 may comprise a single line (e.g., a wire) configured to couple to and/or extend from the sensor device 702. For example, the single line may form a first loop 721a, a second loop 721b, and/or may attach to (e.g., wrap at least partially around) the sensor device 702. Alternatively, the first loop 721a and the second loop 721b may comprise separate lines and/or other components.

The first loop 721a and/or second loop 721b may have any of a variety of suitable shapes. In some examples, the first loop 721a and/or second loop 721b may have variable widths and/or may be configured to extend from a minimal width at or near the sensor device 702 to a maximum and/or greater width at a distal portion 722 of the first loop 721a and/or second loop 721b. For example, a loop 721 may extend into a generally flat and/or curved distal portion 722 as the loop 721 changes direction between attachment points (e.g., two attachment points) between the loop 721 and the sensor device 702.

While the anchoring features 704 are shown in FIG. 7 as forming mushroom-shaped loops extending from either side of the sensor device 702, the anchoring features 704 may have other forms and/or shapes. For example, the one or more anchoring features 704 may comprise generally linear extensions having ends configured to contact and/or press against one or more tissue walls.

In some examples, one or more of the anchoring features 704 may comprise one or more barbs 709, which can include needles, hooks, and/or other extensions, extending from the one or more anchoring features 704 and/or loops 721. The one or more barbs 709 may be configured to penetrate and/or grip onto a surface of a tissue wall. In some examples, the barbs 709 may be configured to extend from multiple sides of the anchoring features 704 and/or may extend at an angle with respect to the anchoring features 704. Barbs 709 may extend from an entire length of a loop 721 and/or may extend from only a portion of a loop 721 (e.g., including the distal portion 722) configured to contact one or more tissue walls.

FIG. 8 provides a side view of a sensor implant device 800 in accordance with one or more examples. The sensor implant device 800 can comprise a sensor assembly/device 802 coupled, attached, and/or otherwise releasably and/or permanently secured to one or more anchoring features 804. The one or more anchoring features can comprise one or more clips, puncture coils, hooks, arms, cords, and/or other features configured for anchoring and/or attachment at one or more areas of tissue within a heart.

The sensor device 802 may comprise at least one sensor component 805. The sensor component(s) 805 may comprise any type of sensor device as described in detail above. The sensor implant device 800 may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 804 may be configured to extend in multiple directions around the sensor device 802 and/or may extend in generally opposite directions laterally (i.e., along a diameter of the sensor device 802) from the sensor device 802. The one or more anchoring features 804 may extend linearly and/or non-linearly away from and/or along the sensor device 802. In some examples, the one or more anchoring features 804 extend longitudinally (e.g., along a shaft of the sensor device 802) along the sensor device 802.

The one or more anchoring features 804 may comprise one or more lines, which can include wires and/or cords, forming one or more loops. In some examples, the one or more loops may be configured to extend across one or more blood flow pathways of a heart and/or to contact one or more tissue walls. The one or more anchoring features 804 may comprise a single line (e.g., a wire) configured to couple to and/or extend from the sensor device 802. For example, the single line may form a first loop 821a, a second loop 821b, and/or may attach to (e.g., wrap at least partially around) the sensor device 802. Alternatively, the first loop 821a and the second loop 821b may comprise separate lines and/or other components.

The first loop 821a and/or second loop 821b may have any of a variety of suitable shapes. In some examples, the first loop 821a and/or second loop 821b may have variable widths and/or may be configured to extend from a minimal width at or near the sensor device 802 to a maximum and/or greater width at a distal portion 822 of the first loop 821a and/or second loop 821b. For example, a loop 821 may extend into a generally flat and/or curved distal portion 822 as the loop 821 changes direction between attachment points (e.g., two attachment points) between the loop 821 and the sensor device 802.

In some examples, the one or more anchoring features 804 may be configured to attach to and/or extend from the sensor device 802 at or near a sensor component 805 of the sensor device 802. For example, a sensor component 805 may be situated at or near a first end of the sensor device 802 and/or the one or more anchoring features 804 may be configured to couple at and/or extend from the first end of the sensor device 802.

The sensor implant device 800 may comprise a housing 813. In some examples, the housing 813 may be configured to secure the one or more anchoring features 804 to the sensor device 802. The housing 813 may be configured to cover at least a portion of the one or more anchoring features 804 and/or may comprise one or more openings to allow the one or more anchoring features 804 to extend through the housing 813. The housing 813 may have a generally cylindrical and/or ring-shaped form. In some examples, the housing 813 may be situated adjacent to and/or near the sensor component 805. The housing 813 and/or the sensor component 805 may be situated at or near a first end of the sensor body 802 and/or the one or more loops may extend from the first end of the sensor body 802.

In some examples, one or more of the anchoring features 804 may comprise one or more barbs 809, which can include needles, hooks, and/or other extensions, extending from the one or more anchoring features 804 and/or loops 821. The one or more barbs 809 may be configured to penetrate and/or grip onto a surface of a tissue wall. In some examples, the barbs 809 may be configured to extend from multiple sides of the anchoring features 804 and/or may extend at an angle with respect to the anchoring features 804. Barbs 809 may extend from an entire length of a loop 821 and/or may extend from only a portion of a loop 821 (e.g., including the distal portion 822) configured to contact one or more tissue walls.

In some examples, at least portions of the one or more anchoring features 804 (e.g., at least the distal portions 822 of the anchoring features 804) may be configured to naturally extend away from each other.

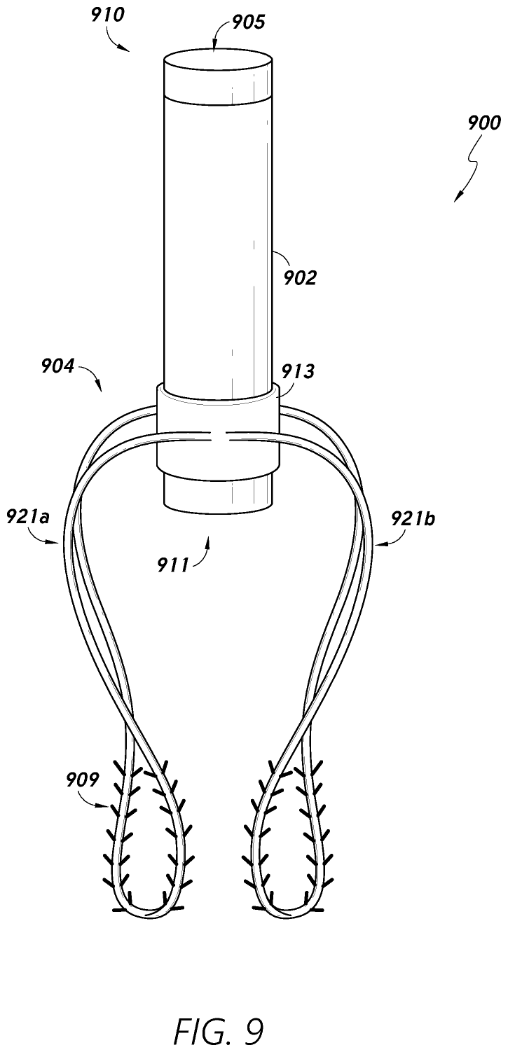

FIG. 9 provides a side view of a sensor implant device 900 in accordance with one or more examples. The sensor implant device 900 can comprise a sensor assembly/device 902 coupled, attached, and/or otherwise releasably and/or permanently secured to one or more anchoring features 904. The one or more anchoring features can comprise one or more clips, puncture coils, hooks, arms, cords, and/or other features configured for anchoring and/or attachment at one or more areas of tissue within a heart.

The sensor device 902 may comprise at least one sensor component 905. The sensor component(s) 905 may comprise any type of sensor device as described in detail above. The sensor implant device 900 may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 904 may be configured to extend in multiple directions around the sensor device 902 and/or may extend in generally opposite directions laterally (i.e., along a diameter of the sensor device 902) from the sensor device 902. The one or more anchoring features 904 may extend linearly and/or non-linearly away from and/or along the sensor device 902. In some examples, the one or more anchoring features 904 extend longitudinally (e.g., along a shaft of the sensor device 902) along the sensor device 902.

The one or more anchoring features 904 may comprise one or more lines, which can include wires and/or cords, forming one or more loops. In some examples, the one or more loops may be configured to extend across one or more blood flow pathways of a heart and/or to contact one or more tissue walls. The one or more anchoring features 904 may comprise a single line (e.g., a wire) configured to couple and/or extend from the sensor device 902. For example, the single line may form a first loop 921a, a second loop 921b, and/or may attach to (e.g., wrap at least partially around) the sensor device 902. Alternatively, the first loop 921a and the second loop 921b may comprise separate lines and/or other components.

In some examples, the one or more anchoring features 904 may be configured to attach to and/or extend from a portion of the sensor device 902 that may be distal from a sensor component 905 of the sensor device 902. For example, a sensor component 905 may be situated at or near a first end 910 of the sensor device 902 and/or the one or more anchoring features 904 may be configured to couple at and/or extend from a second end 911 of the sensor device 902 that is distal and/or opposite from the first end 910 and/or distal from the sensor component 905.

The sensor implant device 900 may comprise a housing 913. In some examples, the housing 913 may be configured to secure the one or more anchoring features 904 to the sensor device 902. The housing 913 may be configured to cover at least a portion of the one or more anchoring features 904 and/or may comprise one or more openings to allow the one or more anchoring features 904 to extend through the housing 913. The housing 913 may have a generally cylindrical and/or ring-shaped form. The sensor component 905 may be situated at a first end of the sensor body 902 and/or the housing 913 may be situated at a second end of the sensor body 902. The one or more loops 921 may be configured to extend from a different end of the device than the sensor component 905 and/or may be configured to extend generally in parallel with the sensor body 902 and/or away from the sensor component 905 and/or the first end of the sensor body 902.

In some examples, one or more of the anchoring features 904 may comprise one or more barbs 909, which can include needles, hooks, and/or other extensions, extending from the one or more anchoring features 904 and/or loops 921. The one or more barbs 909 may be configured to penetrate and/or grip onto a surface of a tissue wall. In some examples, the barbs 909 may be configured to extend from multiple sides of the anchoring features 904 and/or may extend at an angle with respect to the anchoring features 904. Barbs 909 may extend from an entire length of a loop 921 and/or may extend from only a portion of a loop 921 configured to contact one or more tissue walls.

In some examples, at least a portion of the one or more anchoring features 904 (e.g., at least distal portions of the one or more anchoring features 904) may be configured to naturally extend towards each other.

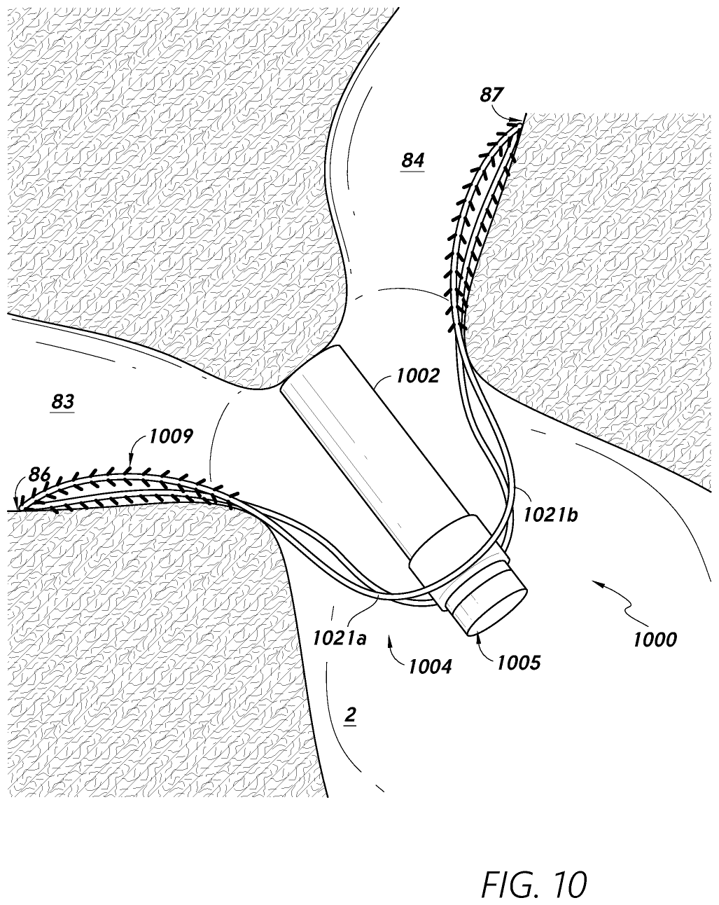

FIG. 10 provides a side view of a sensor implant device 1000 implanted and/or anchored within a heart in accordance with one or more examples. The sensor implant device 1000 can comprise a sensor assembly/device 1002 coupled, attached, and/or otherwise releasably and/or permanently secured to one or more anchoring features 1004. The one or more anchoring features can comprise one or more clips, puncture coils, hooks, arms, cords, and/or other features configured for anchoring and/or attachment at one or more areas of tissue within a heart.

The sensor device 1002 may comprise at least one sensor component 1005. The sensor component(s) 1005 may comprise any type of sensor device as described in detail above. The sensor implant device 1000 may be configured to position the sensor component(s) at a target location within a body, which can include a heart chamber (e.g., a left atrium 2), an opening (e.g., a left atrial appendage) into and/or from a heart chamber, and/or a blood flow pathway (e.g., a coronary sinus).

In some examples, the one or more anchoring features 1004 may be configured to extend in multiple directions around the sensor device 1002 and/or may extend in generally opposite directions laterally (i.e., along a diameter of the sensor device 1002) from the sensor device 1002. The one or more anchoring features 1004 may extend linearly and/or non-linearly away from and/or along the sensor device 1002. In some examples, the one or more anchoring features 1004 extend longitudinally (i.e., along a shaft of the sensor device 1002) along the sensor device 1002.

The one or more anchoring features 1004 may comprise one or more lines, which can include wires and/or cords, forming one or more loops 1021. In some examples, the one or more loops 1021 may be configured to extend across one or more blood flow pathways of a heart and/or to contact one or more tissue walls. The one or more anchoring features 1004 may comprise a single line (e.g., a wire) configured to couple and/or extend from the sensor device 1002. For example, the single line may form a first loop 1021a, a second loop 1021b, and/or may attach to (e.g., wrap at least partially around) the sensor device 1002. Alternatively, the first loop 1021a and the second loop 1021b may comprise separate lines and/or other components.

In the example shown in FIG. 10, the first loop 1021a is situated and/or anchored within a first blood flow pathway (e.g., a first pulmonary vein) 83 and/or the second loop 1021b is situated and/or anchored in a second blood flow pathway 84 (e.g., a second pulmonary vein). The sensor device 1002 and/or at least one sensor component 1005 of the sensor device 1002 may be situated at least partially outside the first blood flow pathway 83 and/or the second blood flow pathway 84 and/or the sensor device 1002 and/or at least one sensor component 1005 of the sensor device 1002 may be situated at least partially within a chamber 2 of a heart (e.g., a left atrium). In some examples, the sensor device 1002 and/or at least one sensor component 1005 of the sensor device 1002 may be situated below, above, and/or near a valve of a heart (e.g., the mitral valve).

The one or more anchoring features 1004 may be configured to extend away from the sensor device 1002 and/or to press against a first distal wall 86 of the first blood flow pathway 83 and/or against a second distal wall 87 of the second blood flow pathway 84. In some examples, the one or more anchoring features 1004 may be shape-set in a pre-determined form to cause the one or more anchoring features 1004 to extend away from the sensor device 1002 and/or each other upon activation and/or removal from various delivery systems. For example, the one or more anchoring features 1004 can comprise a first loop 1021a and/or a second loop 1021b configured to bend and/or collapse against the sensor device 1002 while within a catheter and/or sheath. In response to removal from the catheter and/or sheath and/or activation from one or more pull wires, the first loop 1021a and/or second loop 1021b may be configured to expand to fill space within blood flow pathways and/or to anchor the sensor implant device 1000 at a desired position. In some examples, the one or more anchoring features 1004 may be at least partially composed of one or more shape-memory alloys (e.g., Nitinol) to allow the one or more anchoring features 1004 to be shape-set in a desired form.

In some examples, the sensor implant device 1000 may be configured for transseptal delivery from the right atrium into the left atrium 2. However, the sensor implant device 1000 may be configured for various delivery paths and/or for delivery to various blood pathways and/or chambers within a heart.

The one or more anchoring features 1004 may be configured to position the sensor device 1002 at least partially beyond and/or outside of the first blood flow pathway 83 and/or the second blood flow pathway 84. For example, the one or more anchoring features 1004 may be configured to extend beyond the sensor device 1002 and/or may be configured to extend into the first blood flow pathway 83 and/or the second blood flow pathway 84 while the sensor device 1002 remains at least partially outside the first blood flow pathway 83 and/or the second blood flow pathway 84.

In some examples, one or more of the anchoring features 1004 may comprise one or more barbs 1009, which can include needles, hooks, and/or other extensions, extending from the one or more anchoring features 1004 and/or loops 1021. The one or more barbs 1009 may be configured to penetrate and/or grip onto a surface of a tissue wall. In some examples, the barbs 1009 may be configured to extend from multiple sides of the anchoring features 1004 and/or may extend at an angle with respect to the anchoring features 1004. Barbs 1009 may extend from an entire length of a loop 1021 and/or may extend from only a portion of a loop 1021 configured to contact one or more tissue walls.

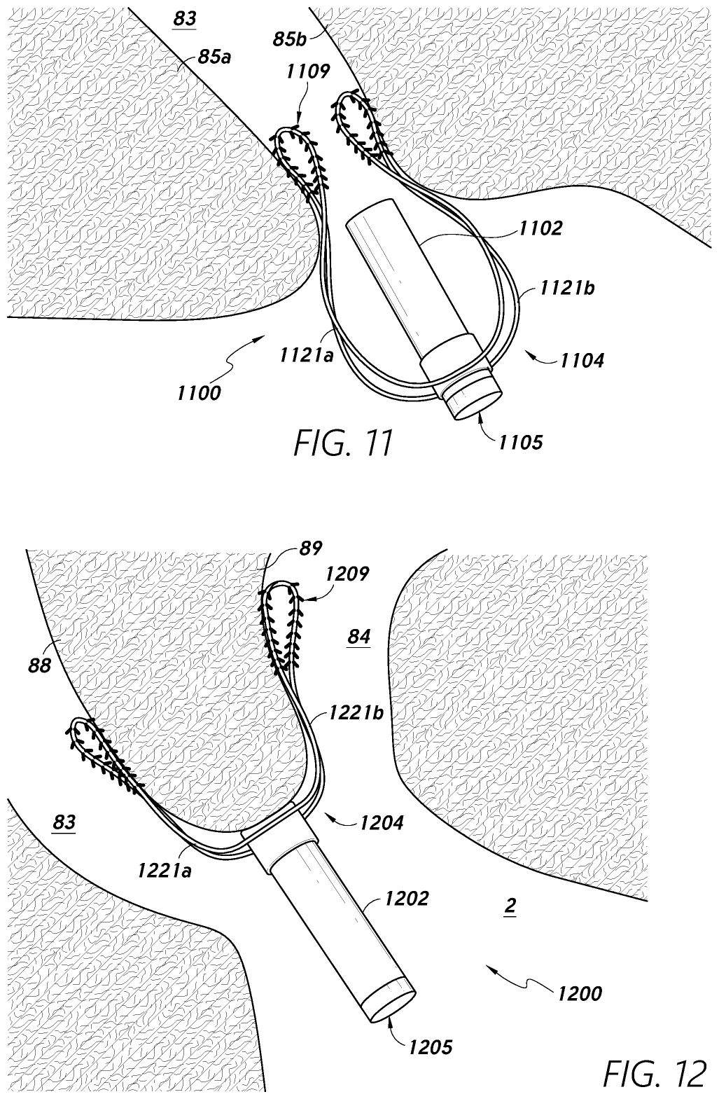

FIG. 11 provides a side view of a sensor implant device 1100 implanted and/or anchored within a heart in accordance with one or more examples. The sensor implant device 1100 can comprise a sensor assembly/device 1102 coupled, attached, and/or otherwise releasably and/or permanently secured to one or more anchoring features 1104. The one or more anchoring features can comprise one or more clips, puncture coils, hooks, arms, cords, and/or other features configured for anchoring and/or attachment at one or more areas of tissue within a heart.

The sensor device 1102 may comprise at least one sensor component 1105. The sensor component(s) 1105 may comprise any type of sensor device as described in detail above. The sensor implant device 1100 may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the one or more anchoring features 1104 may be configured to extend in multiple directions around the sensor device 1102 and/or may extend in generally opposite directions laterally (i.e., along a diameter of the sensor device 1102) from the sensor device 1102. The one or more anchoring features 1104 may extend linearly and/or non-linearly away from and/or along the sensor device 1102 and/or away from each other. In some examples, the one or more anchoring features 1104 extend longitudinally (i.e., along a shaft of the sensor device 1102) along the sensor device 1102.

The one or more anchoring features 1104 may comprise one or more lines, which can include wires and/or cords, forming one or more loops. In some examples, the one or more loops may be configured to extend across one or more blood flow pathways of a heart and/or to contact one or more tissue walls. The one or more anchoring features 1104 may comprise a single line (e.g., a wire) configured to couple and/or extend from the sensor device 1102. For example, the single line may form a first arm 1121a (e.g., loop), a second arm 1121b (e.g., loop), and/or may attach to (e.g., wrap at least partially around) the sensor device 1102. Alternatively, the first arm 1121a and the second arm 1121b may comprise separate lines and/or other components.

In the example shown in FIG. 11, a first arm 1121a and/or a second arm 1121b may be situated and/or anchored within a first blood flow pathway (e.g., a pulmonary vein) 83. The sensor device 1102 and/or at least one sensor component 1105 of the sensor device 1102 may be situated at least partially outside the blood flow pathway 83 and/or the sensor device 1102 and/or at least one sensor component 1105 of the sensor device 1102 may be situated at least partially within a chamber 2 of a heart (e.g., a left atrium). In some examples, the sensor device 1102 and/or at least one sensor component 1105 of the sensor device 1102 may be situated below, above, and/or near a valve of a heart (e.g., the mitral valve).

The one or more anchoring features 1104 may be configured to extend away from the sensor device 1102 and/or to press against a first wall 85a and/or against a second wall 85b of the blood flow pathway 83. In some examples, the one or more anchoring features 1104 may be shape-set in a pre-determined form to cause the one or more anchoring features 1104 to extend away from the sensor device 1102 upon activation and/or removal from various delivery systems. For example, the one or more anchoring features 1104 can comprise a first arm 1121a and/or a second arm 1121b configured to bend and/or collapse against the sensor device 1102 while within a catheter and/or sheath. In response to removal from the catheter and/or sheath and/or activation from one or more pull wires, the first arm 1121a and/or second arm 1121b may be configured to expand to fill space within blood flow pathways and/or to anchor the sensor implant device 1100 at a desired position. In some examples, the one or more anchoring features 1104 may be at least partially composed of one or more shape-memory alloys (e.g., Nitinol) to allow the one or more anchoring features 1104 to be shape-set in a desired form.

In some examples, the sensor implant device 1100 may be configured for transseptal delivery from the right atrium into the left atrium. However, the sensor implant device 1100 may be configured for various delivery paths and/or for delivery to various blood pathways and/or chambers within a heart.

The one or more anchoring features 1104 may be configured to position the sensor device 1102 at least partially beyond and/or outside of the blood flow pathway 83. For example, the one or more anchoring features 1104 may be configured to extend beyond the sensor device 1102 and/or may be configured to extend into the blood flow pathway 83 while the sensor device 1102 remains at least partially outside the blood flow pathway 83.

In some examples, one or more of the anchoring features 1104 may comprise one or more barbs 1109, which can include needles, hooks, and/or other extensions, extending from the one or more anchoring features 1104 and/or loops 1121. The one or more barbs 1109 may be configured to penetrate and/or grip onto a surface of a tissue wall. In some examples, the barbs 1109 may be configured to extend from multiple sides of the anchoring features 1104 and/or may extend at an angle with respect to the anchoring features 1104. Barbs 1109 may extend from an entire length of a loop 1121 and/or may extend from only a portion of a loop 1121 configured to contact one or more tissue walls.

FIG. 12 provides a side view of a sensor implant device 1200 implanted and/or anchored within a heart in accordance with one or more examples. The sensor implant device 1200 can comprise a sensor assembly/device 1202 coupled, attached, and/or otherwise releasably and/or permanently secured to one or more anchoring features 1204. The one or more anchoring features can comprise one or more clips, puncture coils, hooks, arms, cords, and/or other features configured for anchoring and/or attachment at one or more areas of tissue within a heart.

The sensor device 1202 may comprise at least one sensor component 1205. The sensor component(s) 1205 may comprise any type of sensor device as described in detail above. The sensor implant device 1200 may be configured to position the sensor component(s) at a target location within a body, which can include a heart chamber (e.g., a left atrium 2), an opening (e.g., a left atrial appendage) into and/or from a heart chamber, and/or a blood flow pathway (e.g., a coronary sinus).

In some examples, the one or more anchoring features 1204 may be configured to extend in multiple directions around the sensor device 1202 and/or may extend in generally opposite directions laterally (i.e., along a diameter of the sensor device 1202) from the sensor device 1202. The one or more anchoring features 1204 may extend linearly and/or non-linearly away from and/or along the sensor device 1202. In some examples, the one or more anchoring features 1204 extend longitudinally (i.e., along a shaft of the sensor device 1202) along the sensor device 1202.

The one or more anchoring features 1204 may comprise one or more lines, which can include wires and/or cords, forming one or more loops. In some examples, the one or more loops may be configured to extend across one or more blood flow pathways of a heart and/or to contact one or more tissue walls. The one or more anchoring features 1204 may comprise a single line (e.g., a wire) configured to couple and/or extend from the sensor device 1202. For example, the single line may form a first loop 1221a (e.g., arm), a second loop 1221b (e.g., arm), and/or may attach to (e.g., wrap at least partially around) the sensor device 1202. Alternatively, the first loop 1221a and the second loop 1221b may comprise separate lines and/or other components.

In the example shown in FIG. 12, the first loop 1221a is situated and/or anchored within a first blood flow pathway (e.g., a first pulmonary vein) 83 and/or the second loop 1221b is situated and/or anchored in a second blood flow pathway 84 (e.g., a second pulmonary vein). The sensor device 1202 and/or at least one sensor component 1205 of the sensor device 1202 may be situated at least partially outside the first blood flow pathway 83 and/or the second blood flow pathway 84 and/or the sensor device 1202 and/or at least one sensor component 1205 of the sensor device 1202 may be situated at least partially within a chamber 2 of a heart (e.g., a left atrium). In some examples, the sensor device 1202 and/or at least one sensor component 1205 of the sensor device 1202 may be situated below, above, and/or near a valve of a heart (e.g., the mitral valve).

At least a portion of the one or more anchoring features 1204 (e.g., at least generally flat distal portions of the one or more anchoring features 1204) may be configured to extend towards each other and/or to press against a first proximal wall 88 of the first blood flow pathway 83 and/or against a second proximal wall 89 of the second blood flow pathway 84. In some examples, the one or more anchoring features 1204 may be shape-set in a pre-determined form to cause the one or more anchoring features 1204 to extend away from the sensor device 1202 upon activation and/or removal from various delivery systems. For example, the one or more anchoring features 1204 can comprise a first loop 1221a and/or a second loop 1221b configured to bend and/or collapse against the sensor device 1202 while within a catheter and/or sheath. In response to removal from the catheter and/or sheath and/or activation from one or more pull wires, the first loop 1221a and/or second loop 1221b may be configured to expand to fill space within blood flow pathways and/or to anchor the sensor implant device 1200 at a desired position. In some examples, the one or more anchoring features 1204 may be at least partially composed of one or more shape-memory alloys (e.g., Nitinol) to allow the one or more anchoring features 1204 to be shape-set in a desired form.

In some examples, the sensor implant device 1200 may be configured for transseptal delivery from the right atrium into the left atrium 2. However, the sensor implant device 1200 may be configured for various delivery paths and/or for delivery to various blood pathways and/or chambers within a heart.

The one or more anchoring features 1204 may be configured to position the sensor device 1202 at least partially beyond and/or outside of the first blood flow pathway 83 and/or the second blood flow pathway 84. For example, the one or more anchoring features 1204 may be configured to extend beyond the sensor device 1202 and/or may be configured to extend into the first blood flow pathway 83 and/or the second blood flow pathway 84 while the sensor device 1202 remains at least partially outside the first blood flow pathway 83 and/or the second blood flow pathway 84.

In some examples, one or more of the anchoring features 1204 may comprise one or more barbs 1209, which can include needles, hooks, and/or other extensions, extending from the one or more anchoring features 1204 and/or loops 1221. The one or more barbs 1209 may be configured to penetrate and/or grip onto a surface of a tissue wall. In some examples, the barbs 1209 may be configured to extend from multiple sides of the anchoring features 1204 and/or may extend at an angle with respect to the anchoring features 1204. Barbs 1209 may extend from an entire length of a loop 1221 and/or may extend from only a portion of a loop 1221 configured to contact one or more tissue walls.

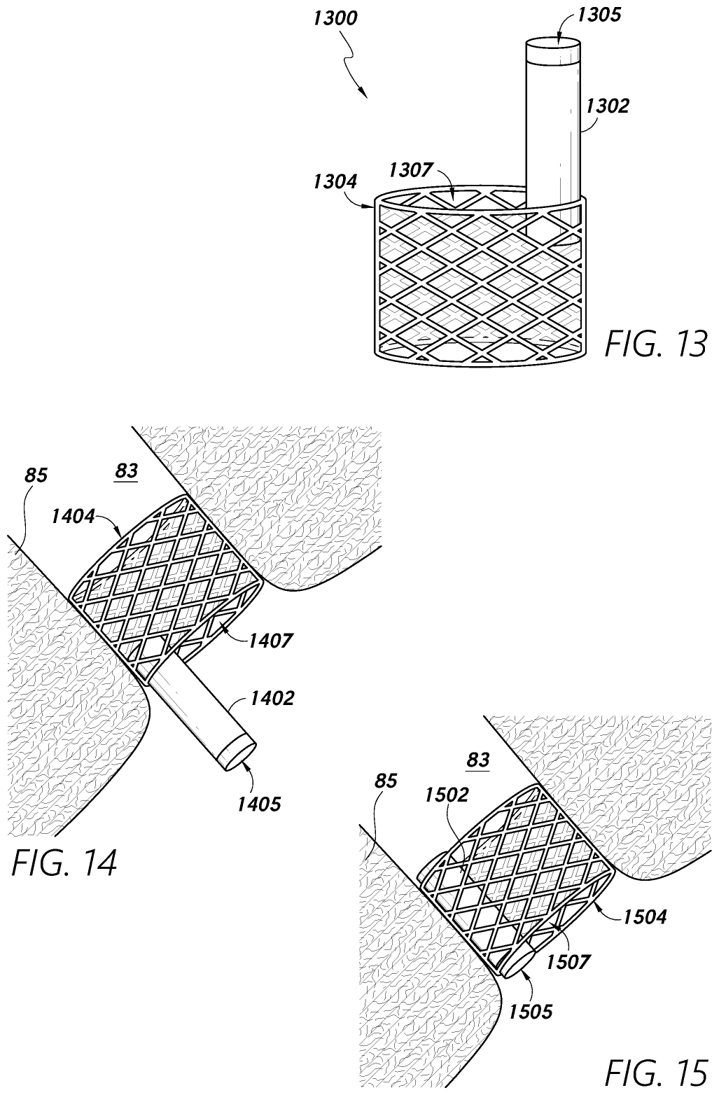

FIG. 13 provides a side view of a sensor implant device 1300 in accordance with one or more examples. The sensor implant device 1300 can comprise a sensor assembly/device 1302 coupled, attached, and/or otherwise releasably and/or permanently secured to a shunt body 1304 and/or other anchoring feature(s). In some examples, the shunt body 1304 can comprise a generally tubular and/or cylindrical frame having an inner lumen 1307 to allow blood flow through the shunt body 1304. The shunt body 1304 may comprise a network of one or more wires and/or cords forming one or more cells having diamond and/or other shapes.

The sensor device 1302 may be configured to attach and/or couple to an inner and/or outer wall of the shunt body 1304. For example, the sensor device 1302 may be situated at least partially within an inner lumen 1307 of the shunt body 1304. The shunt body 1304 may be configured for placement at least partially within one or more blood flow pathways of a heart. At least a portion of the sensor device 1302 (e.g., at least a sensor component 1305 of the sensor device 1302) may extend beyond the inner lumen 1307 of the shunt body 1304.

FIG. 14 provides a side view of a sensor implant device implanted and/or anchored within a heart in accordance with one or more examples. The sensor implant device can comprise a sensor body/device 1402 coupled, attached, and/or otherwise releasably and/or permanently secured to a stent 1404 and/or other anchoring feature(s). In some examples, the sensor device 1402 may have a generally tubular and/or cylindrical form and/or may form an inner lumen 1407. The sensor device 1402 may be configured to couple to an inner wall of the sensor device 1402 and/or the sensor device 1402 may be configured to be situated at least partially within the inner lumen 1407 of the stent 1404. In some examples, at least a portion of the sensor device 1402 (e.g., at least a portion of a sensor component 1405 of the sensor device 1402) may be configured to extend out of and/or beyond the inner lumen 1407 of the stent 1404. In some examples, most of the sensor body 1402 may be situated outside the lumen 1407 of the stent 1404.

In some examples, the stent 1404 may be configured for anchoring within a blood flow pathway 83 of a heart. The stent 1404 may have an at least partially compressible and/or expandable structure to facilitate delivery via one or more delivery devices (e.g., a catheter) and/or to facilitate anchoring within the blood flow pathway 83. For example, the stent 1404 may be configured to assume a compressed profile while within a catheter and/or may be configured to expand in multiple directions in response to removal from the catheter and/or may press against an inner wall 85 of the blood flow pathway 83.

The sensor device 1402 may comprise at least one sensor component 1405. The sensor component(s) 1405 may comprise any type of sensor device as described in detail above. The sensor implant device may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the sensor implant device may be configured for transseptal delivery from the right atrium into the left atrium. However, the sensor implant device may be configured for various delivery paths and/or for delivery to various blood pathways and/or chambers within a heart.

The stent 1404 may be configured to position the sensor device 1402 at least partially beyond and/or outside of the blood flow pathway 83. For example, the stent 1404 may be configured to extend beyond the sensor device 1402 and/or may be configured to extend into the blood flow pathway 83 while the sensor device 1402 remains at least partially outside the blood flow pathway 83.

FIG. 15 provides a side view of a sensor implant device implanted and/or anchored within a heart in accordance with one or more examples. The sensor implant device can comprise a sensor body/device 1502 coupled, attached, and/or otherwise releasably and/or permanently secured to a stent 1504 and/or other anchoring feature(s). In some examples, the sensor device 1502 may have a generally tubular and/or cylindrical form and/or may form an inner lumen 1507. The sensor device 1502 may be configured to couple to an inner wall of the sensor device 1502 and/or the sensor device 1502 may be configured to be situated at least partially within the inner lumen 1507 of the stent 1504. In some examples, the entire sensor device 1502 may be configured to be situated within the inner lumen 1507 of the stent 1504. In some examples, most of the sensor body 1502 may be situated within the lumen 1507 of the stent 1504.

In some examples, the stent 1504 may be configured for anchoring within a blood flow pathway 83 of a heart. The stent 1504 may have an at least partially compressible and/or expandable structure to facilitate delivery via one or more delivery devices (e.g., a catheter) and/or to facilitate anchoring within the blood flow pathway 83. For example, the stent 1504 may be configured to assume a compressed profile while within a catheter and/or may be configured to expand in multiple directions in response to removal from the catheter and/or may press against an inner wall 85 of the blood flow pathway 83.

The sensor device 1502 may comprise at least one sensor component 1505. The sensor component(s) 1505 may comprise any type of sensor device as described in detail above. The sensor implant device may be configured to position the sensor component(s) at a target location within a body, which can include a heart chamber (e.g., a left atrium), an opening (e.g., a left atrial appendage) into and/or from a heart chamber, and/or a blood flow pathway (e.g., a coronary sinus).

In some examples, the sensor implant device may be configured for transseptal delivery from the right atrium into the left atrium. However, the sensor implant device may be configured for various delivery paths and/or for delivery to various blood pathways and/or chambers within a heart.

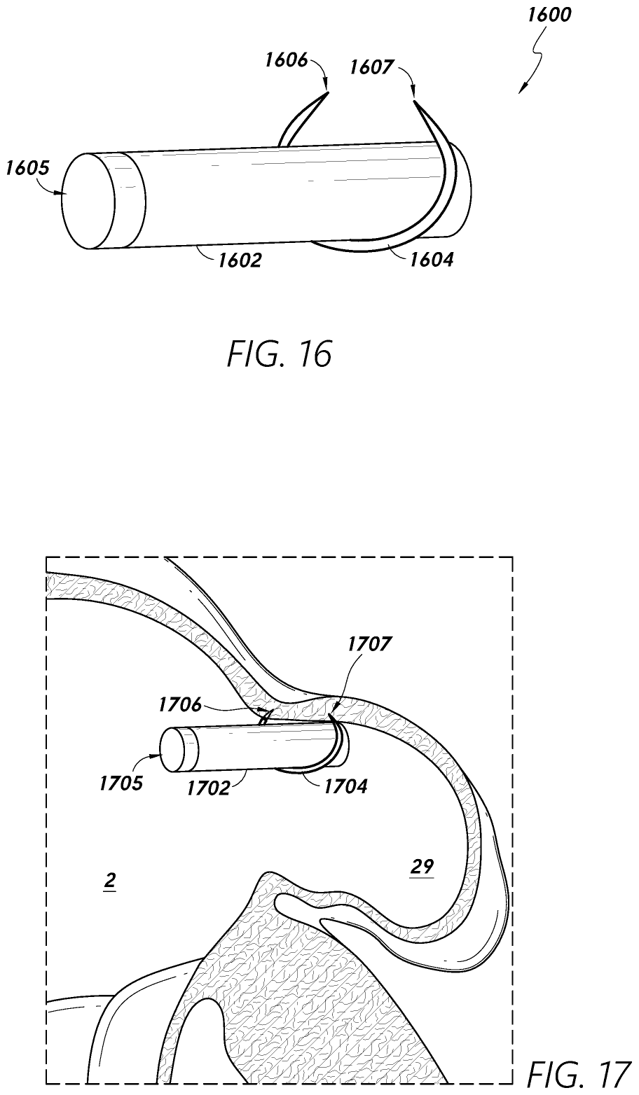

FIG. 16 illustrates a sensor implant device 1600 in accordance with one or more examples. The sensor implant device 1600 can comprise a sensor assembly/device 1602 coupled, attached, and/or otherwise releasably and/or permanently secured to a C-shaped and/or generally curved clip 1604 and/or anchoring feature(s). In some examples, the clip 1604 may comprise a first end 1606 and/or a second end 1607 configured to pierce and/or otherwise anchor to one or more tissue walls. The clip 1604 may be configured to wrap at least partially around and/or otherwise secure to the sensor device 1602. While the clip 1604 is shown as a single device, the clip 1604 may comprise multiple segments. For example, the first end 1606 and the second end 1607 may be separate components and/or may be configured to separately couple to the sensor device 1602. The sensor device 1602 may comprise one or more sensor components 1605.

In some examples, the clip 1604 may have shape-memory properties and/or may be biased to a closed configuration in which the first end 1606 and the second end 1607 are positioned close together. For example, the clip 1604 may be at least partially composed of Nitinol and/or one or more other shape-memory alloys and/or materials. The clip 1604 may be at least partially flexible and/or elastic such that the first end 1606 and/or the second end 1607 may be pulled apart to fit a portion of tissue between the first end 1606 and the second end 1607. In some examples, pull wires and/or other systems may be attached to the clip 1604 and/or may otherwise be used to cause movement of the first end 1606 and/or the second end 1607. In response to removal of pulling force, the first end 1606 and the second end 1607 may be configured to move closer together in a pincer grip manner to cause the first end 1606 and/or the second end 1607 to puncture, grasp, and/or otherwise anchor to one or more areas of tissue.

FIG. 17 illustrates a sensor implant device implanted and/or anchored within a heart in accordance with one or more examples. The sensor implant device is shown anchored in a left atrial appendage. However, the sensor implant device may be configured for anchoring at other areas of tissue.

The sensor implant device can comprise a sensor assembly/device 1702 coupled, attached, and/or otherwise releasably and/or permanently secured to a clip 1704 and/or anchoring feature(s). In some examples, the clip 1704 may comprise a first end 1706 and/or a second end 1707 configured to pierce and/or otherwise anchor to one or more tissue walls. The clip 1704 may be configured to wrap at least partially around and/or otherwise secure to the sensor device 1702. While the clip 1704 is shown as a single device, the clip 1704 may comprise multiple segments. For example, the first end 1706 and the second end 1707 may be separate components and/or may be configured to separately couple to the sensor device 1702.

In some examples, the clip 1704 may have shape-memory properties and/or may be biased to a closed configuration in which the first end 1706 and the second end 1707 are positioned close together. For example, the clip 1704 may be at least partially composed of Nitinol and/or one or more other shape-memory alloys and/or materials. The clip 1704 may be at least partially flexible and/or elastic such that the first end 1706 and/or the second end 1707 may be pulled apart to fit a portion of tissue between the first end 1706 and the second end 1707. In some examples, pull wires and/or other systems may be attached to the clip 1704 and/or may otherwise be used to cause movement of the first end 1706 and/or the second end 1707. In response to removal of pulling force, the first end 1706 and the second end 1707 may be configured to move closer together in a pincer grip manner to cause the first end 1706 and/or the second end 1707 to puncture, grasp, and/or otherwise anchor to one or more areas of tissue.

The sensor device 1702 may comprise at least one sensor component 1705. The sensor component(s) 1705 may comprise any type of sensor device as described in detail above. The sensor implant device may be configured to position the sensor component(s) 1705 at a target location within a body, which can include a heart chamber (e.g., a left atrium 2), an opening (e.g., a left atrial appendage 29) into and/or from a heart chamber, and/or a blood flow pathway (e.g., a coronary sinus). In some examples, the sensor implant device may be configured to position at least the sensor component(s) outside of the left atrial appendage.

In some examples, the sensor implant device may be configured for transseptal delivery from the right atrium into the left atrium 2. However, the sensor implant device may be configured for various delivery paths and/or for delivery to various blood pathways and/or chambers within a heart.

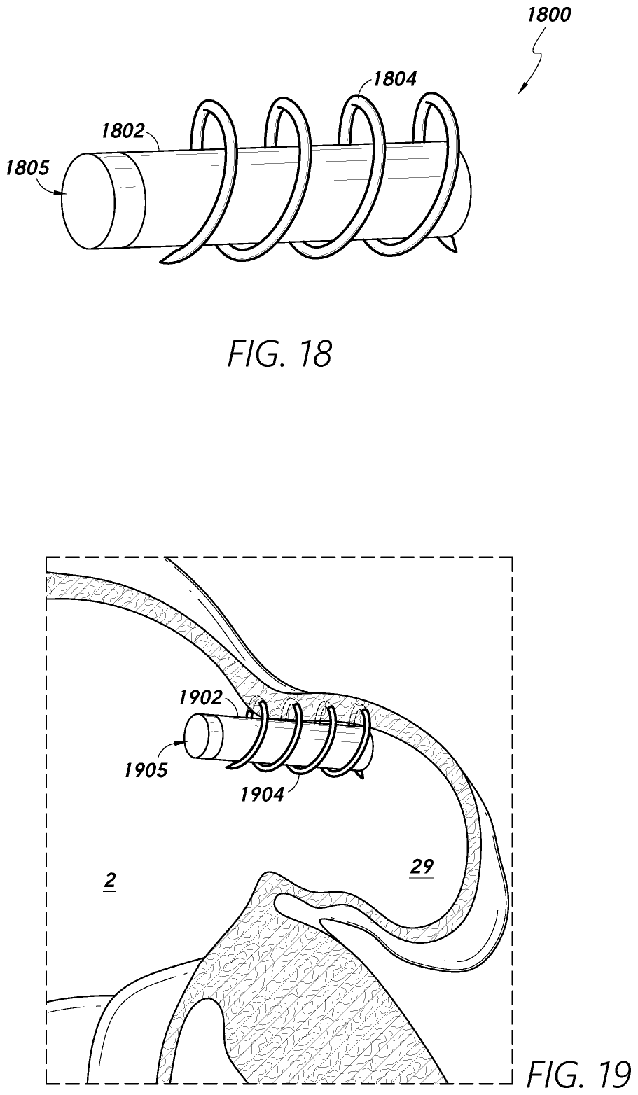

FIG. 18 illustrates a sensor implant device 1800 in accordance with one or more examples. The sensor implant device 1800 can comprise a sensor assembly/device 1802 coupled, attached, and/or otherwise releasably and/or permanently secured to a coil 1804 and/or anchoring feature(s). In some examples, the coil 1804 may comprise one or more winds configured to at least partially encircle and/or wrap at least partially around the sensor device 1802 and/or to pierce and/or otherwise anchor to one or more portions of a tissue wall. The coil 1804 may comprise a single wire forming multiple winds and/or may comprise multiple circular rings which may or may not be interconnected. The sensor device 1802 may be configured to fit at least partially within an inner lumen formed by the coil 1804. In some examples, the sensor device 1802 may be held in place by friction between the sensor device 1802 and the winds of the coil 1804. Additionally or alternatively, the sensor device 1802 may be coupled to and/or secured to the coil 1804.

In some examples, the coil 1804 may have shape-memory properties and/or may be configured to naturally assume the form shown in FIG. 18 in response to removal of outside forces. The coil 1804 may be configured to be straightened and/or compressed to fit within a delivery device (e.g., a catheter). Upon removal from the catheter, the coil 1804 may be configured to form one or more generally circular winds. At least a portion of the sensor device 1802 (e.g., at least a sensor component 1805 of the sensor device 1802) may be configured to extend out of and/or beyond the inner lumen of the coil 1804.

FIG. 19 illustrates a sensor implant device implanted and/or anchored within a heart in accordance with one or more examples. The sensor implant device is shown anchored in a left atrial appendage 29. However, the sensor implant device may be configured for anchoring at other areas of tissue.

The sensor implant device can comprise a sensor assembly/device 1902 coupled, attached, and/or otherwise releasably and/or permanently secured to a coil 1904 and/or anchoring feature(s). In some examples, the coil 1904 may comprise one or more winds configured to wrap at least partially around the sensor device 1902 and/or to pierce and/or otherwise anchor to a tissue wall. The coil 1904 may comprise a single wire forming multiple winds and/or may comprise multiple circular rings which may or may not be interconnected and/or may at least partially encircle the sensor device 1902. The sensor device 1902 may be configured to fit at least partially within an inner lumen formed by the coil 1904. In some examples, the sensor device 1902 may be held in place by friction between the sensor device 1902 and the winds of the coil 1904. Additionally or alternatively, the sensor device 1902 may be coupled to and/or secured to the coil 1904.

In some examples, the coil 1904 may have shape-memory properties and/or may be configured to naturally assume the form shown in FIG. 19 in response to removal of outside forces. The coil 1904 may be configured to be straightened and/or compressed to fit within a delivery device (e.g., a catheter). Upon removal from the catheter, the coil 1904 may be configured to form one or more generally circular winds.

The coil 1904 may be configured to puncture and/or anchor to an area of tissue at multiple points. For example, the coil 1904 may be twisted such that a first end of the coil 1904 punctures multiple portions of tissue in a generally linear orientation. The coil 1904 may comprise a pointed tip configured to penetrate the tissue at one or more points.

The sensor device 1902 may comprise at least one sensor component 1905. The sensor component(s) 1905 may comprise any type of sensor device as described in detail above. The sensor implant device may be configured to position the sensor component(s) 1905 at a target location within a body, which can include a heart chamber (e.g., a left atrium 2), an opening (e.g., a left atrial appendage 29) into and/or from a heart chamber, and/or a blood flow pathway (e.g., a coronary sinus). In some examples, the sensor implant device may be configured to position at least the sensor component(s) 1905 outside of the left atrial appendage 29.

In some examples, the sensor implant device 1902 may be configured for transseptal delivery from the right atrium into the left atrium 2. However, the sensor implant device 1902 may be configured for various delivery paths and/or for delivery to various blood pathways and/or chambers within a heart.

The coil 1904 may be configured to position the sensor device 1902 at least partially beyond and/or outside of the left atrial appendage 29. For example, the coil 1904 may be configured to extend beyond the sensor device 1902 and/or may be configured to extend into the left atrial appendage 29 while the sensor device 1902 remains at least partially outside the left atrial appendage 29.

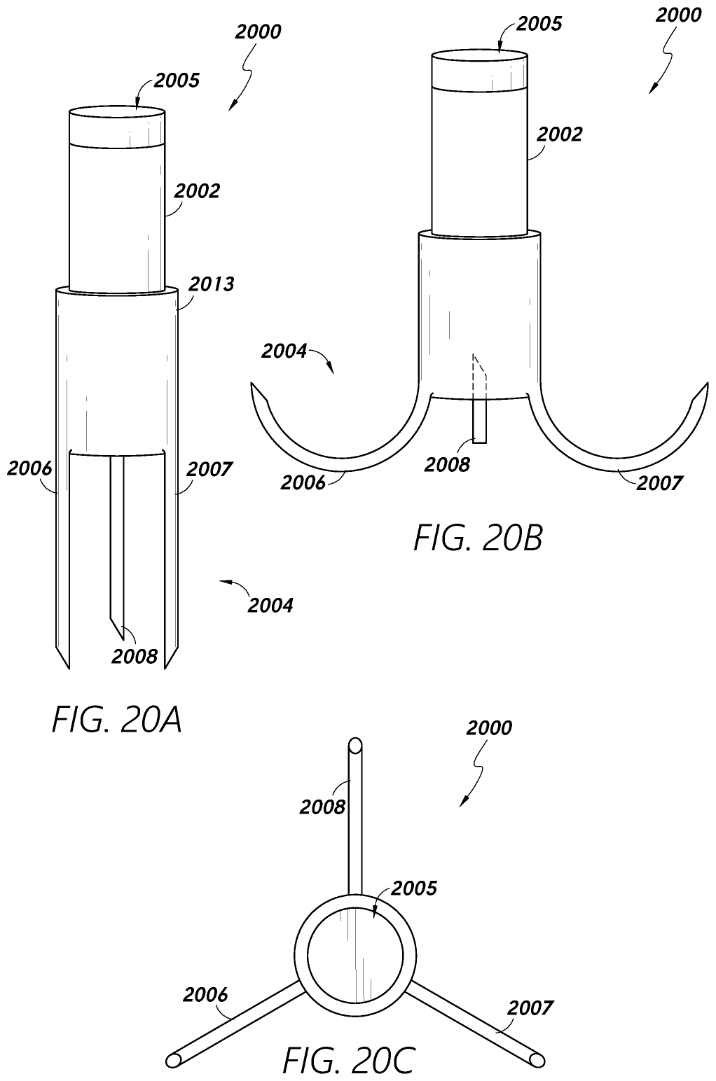

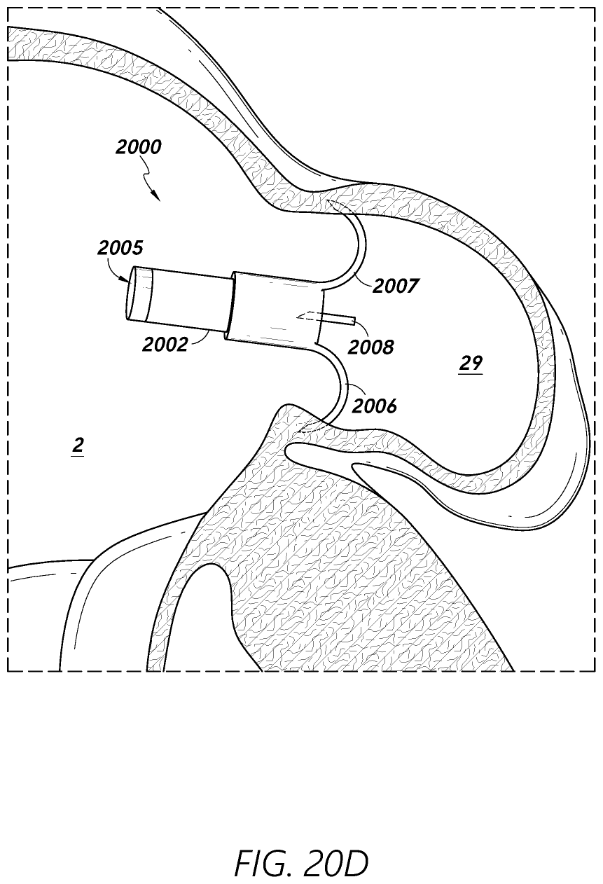

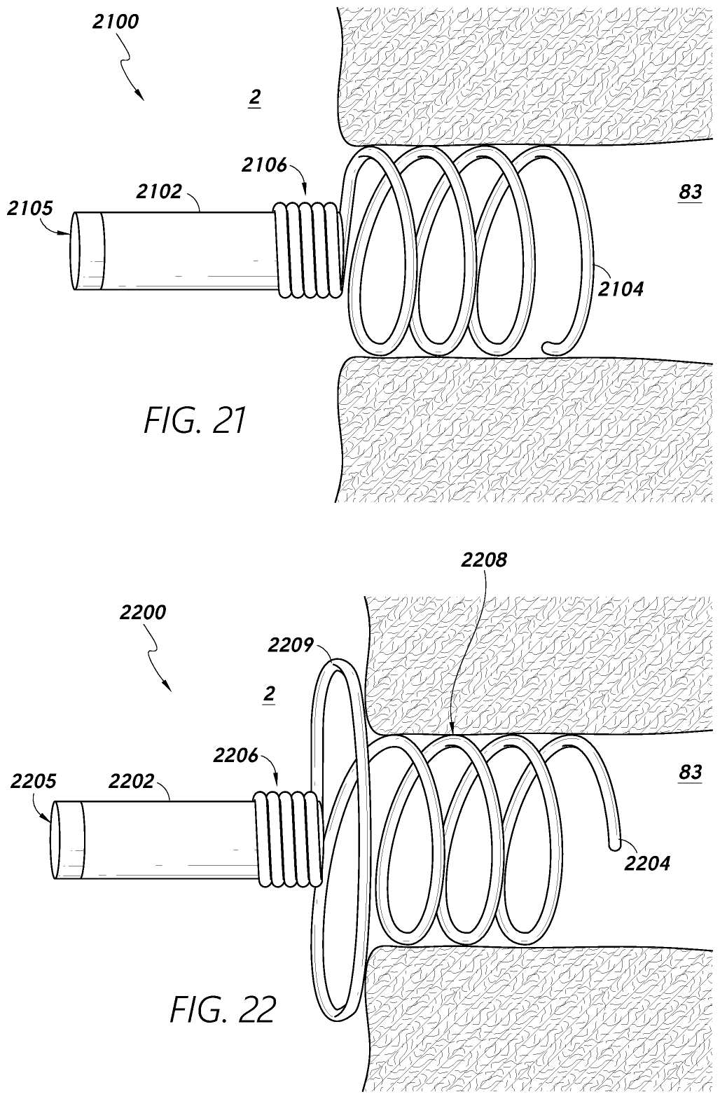

FIGS. 20A-20D illustrate a sensor implant device 2000 in accordance with one or more examples. The sensor implant device 2000 can comprise a sensor assembly/device 2002 coupled, attached, and/or otherwise releasably and/or permanently secured to one or more anchoring features 2004. In some examples, the one or more anchoring features 2004 may comprise one or more arms and/or hooks which can include a first arm 2006, a second arm 2007, and/or a third arm 2008 configured to pierce and/or otherwise anchor to one or more tissue walls.

In some examples, the anchoring features 2004 may have shape-memory properties and/or may be biased to an expanded configuration in which the first arm 2006, second arm 2007, and/or third arm 2008 may assume a generally curved and/or “fishhook” configuration, as shown in FIG. 20B. For example, the anchoring features 2004 may be at least partially composed of Nitinol and/or one or more other shape-memory alloys and/or materials. The anchoring features 2004 may be at least partially flexible and/or elastic such that the first arm 2006, second arm 2007, and/or third arm 2008 may be straightened to a generally linear and/or parallel configuration, as shown in FIG. 20A. For example, the first arm 2006, second arm 2007, and/or third arm 2008 may be oriented generally in-line and/or parallel with a length of the sensor device 2002. The first arm 2006, second arm 2007, and/or third arm 2008 may be configured to assume the linear configuration shown in FIG. 20A during delivery (e.g., while within a catheter and/or other delivery device) and/or may be configured to assume the curved/expanded configuration shown in FIG. 20B after delivery (e.g., upon removal from a catheter). In some examples, the first arm 2006, second arm 2007, and/or third arm 2008 may be configured to naturally expand and/or extend/curve away from and/or perpendicularly to the sensor device 2002 in response to removal of the first arm 2006, second arm 2007, and/or third arm 2008 from the catheter and/or other delivery device. The first arm 2006, second arm 2007, and/or third arm 2008 may be configured to extend at least partially away from each other in response to removal from the catheter. FIG. 20C provides an overhead view of the sensor implant device 2000 in the expanded configuration.

The sensor implant device 2000 may comprise a housing 2013. In some examples, the housing 2013 may be configured to secure the one or more anchoring features 2004 to the sensor device 2002. The housing 2013 may be configured to cover at least a portion of the one or more anchoring features 2004 and/or may comprise one or more openings to allow the one or more anchoring features 2004 to extend through the housing 2013. The housing 2013 may have a generally cylindrical and/or ring-shaped form.

FIG. 20D illustrates the sensor implant device 2000 implanted and/or anchored within a heart in accordance with one or more examples. The sensor implant device 2000 is shown anchored in a left atrial appendage 29. However, the sensor implant device 2000 may be configured for anchoring at other areas of tissue.

The sensor implant device 2000 can comprise a sensor assembly/device 2002 coupled, attached, and/or otherwise releasably and/or permanently secured to one or more anchoring features 2004, which can include a first arm 2006, second arm 2007, and/or third arm 2008.

The sensor device 2002 may comprise at least one sensor component 2005. The sensor component(s) 2005 may comprise any type of sensor device as described in detail above. The sensor implant device 2000 may be configured to position the sensor component(s) 2005 at a target location within a body, which can include a heart chamber (e.g., a left atrium 2), an opening (e.g., a left atrial appendage 29) into and/or from a heart chamber, and/or a blood flow pathway (e.g., a coronary sinus). In some examples, the sensor implant device 2000 may be configured to position at least the sensor component(s) 2005 outside the left atrial appendage 29.

In some examples, the sensor implant device 2000 may be configured for transseptal delivery from the right atrium into the left atrium 2. However, the sensor implant device 2000 may be configured for various delivery paths and/or for delivery to various blood pathways and/or chambers within a heart.

The one or more anchoring features 2004 may be configured to position the sensor device 2002 at least partially beyond and/or outside of the left atrial appendage 29. For example, the one or more anchoring features 2004 may be configured to extend beyond the sensor device 2002 and/or may be configured to extend into the left atrial appendage 29 while the sensor device 2002 remains at least partially outside the left atrial appendage 29.

FIG. 21 illustrates a sensor implant device 2100 in accordance with one or more examples. The sensor implant device 2100 can comprise a sensor assembly/device 2102 coupled, attached, and/or otherwise releasably and/or permanently secured to a coil 2104 and/or anchoring feature(s). In some examples, the coil 2104 may comprise one or more winds configured to wrap at least partially around the sensor device 2102 and/or to pierce and/or otherwise anchor to a tissue wall. The coil 2104 may comprise a single wire forming multiple winds. The sensor device 2102 may be configured to fit at least partially within an inner lumen formed by the coil 2104 and/or to extend from a first end 2106 of the coil 2104 and/or at least partially out of and/or beyond the inner lumen of the coil 2104. In some examples, the sensor device 2102 may be held in place by friction between the sensor device 2102 and the winds of the coil 2104. Additionally or alternatively, the sensor device 2102 may be coupled to and/or secured to the coil 2104. For example, the first end 2106 may be configured to form one or winds around the sensor device 2102 to securely couple to the sensor device 2102.

In some examples, the coil 2104 may have shape-memory properties and/or may be configured to naturally assume the form shown in FIG. 21 in response to removal of outside forces. The coil 2104 may be configured to be straightened and/or compressed to fit within a delivery device (e.g., a catheter). Upon removal from the catheter, the coil 2104 may be configured to form one or more generally circular winds. In some examples, the coil 2104 may be configured to expand in diameter until the coil 2104 presses against inner walls of a blood flow pathway 83 to hold the coil 2104 within the blood flow pathway 83. Additionally or alternatively, the sensor implant device 2100 may be anchored in a left atrial appendage and/or other area(s) of tissue.

The sensor device 2102 may comprise at least one sensor component 2105. The sensor component(s) 2105 may comprise any type of sensor device as described in detail above. The sensor implant device 2100 may be configured to position the sensor component(s) 2105 at a target location within a body, which can include a heart chamber (e.g., a left atrium 2), an opening (e.g., a left atrial appendage) into and/or from a heart chamber, and/or a blood flow pathway 83 (e.g., a coronary sinus). In some examples, the sensor implant device 2100 may be configured to position at least the sensor component(s) outside of the blood flow pathway 83.

In some examples, the sensor implant device 2100 may be configured for transseptal delivery from the right atrium into the left atrium 2. However, the sensor implant device 2100 may be configured for various delivery paths and/or for delivery to various blood pathways 83 and/or chambers within a heart.

The coil 2104 may be configured to position the sensor device 2102 at least partially beyond and/or outside of the blood flow pathway 83. For example, the coil 2104 may be configured to extend beyond the sensor device 2102 and/or may be configured to extend into the blood flow pathway 83 while the sensor device 2102 remains at least partially outside the blood flow pathway 83.

FIG. 22 illustrates a sensor implant device 2200 in accordance with one or more examples. The sensor implant device 2200 can comprise a sensor assembly/device 2202 coupled, attached, and/or otherwise releasably and/or permanently secured to a coil 2204 and/or anchoring feature(s). In some examples, the coil 2204 may comprise one or more winds configured to wrap at least partially around the sensor device 2202 and/or to pierce and/or otherwise anchor to a tissue wall. The coil 2204 may comprise a single wire forming multiple winds. In some examples, only a portion of the coil 2204 (e.g., a first/distal portion 2208) may be configured for placement with a blood flow pathway 83. For example, the coil 2204 may comprise a proximal portion 2209 (e.g., situated proximal to the sensor device 2202) having a greater width and/or diameter than other portions (e.g., the first portion 2208) of the coil 2204. The first portion 2208 of the coil 2204 may be configured to anchor in the blood flow pathway 83 by expanding to press against the inner surface of the blood flow pathway 83, left atrial appendage, and/or other area of tissue.

The sensor device 2202 may be configured to fit at least partially within an inner lumen formed by the coil 2204 and/or to extend from a first end 2206 of the coil 2204 and/or at least partially out of and/or beyond the inner lumen of the coil 2204. In some examples, the sensor device 2202 may be held in place by friction between the sensor device 2202 and the winds of the coil 2204. Additionally or alternatively, the sensor device 2202 may be coupled to and/or secured to the coil 2204. For example, the first end 2206 may be configured to form one or winds around the sensor device 2202 to securely couple to the sensor device 2202. In some examples, the coil 2204 may have a variable diameter and/or may comprise a proximal portion 2209 configured to expand to a greater width and/or diameter than a blood flow pathway 83 and/or a portion of the coil (e.g., a first portion 2208) that is situated, anchored, and/or configured for anchoring in the blood flow pathway 83.

In some examples, the coil 2204 may have shape-memory properties and/or may be configured to naturally assume the form shown in FIG. 22 in response to removal of outside forces. The coil 2204 may be configured to be straightened and/or compressed to fit within a delivery device (e.g., a catheter). Upon removal from the catheter, the coil 2204 may be configured to form one or more generally circular winds. In some examples, the coil 2204 may be configured to expand in diameter until the coil 2204 presses against inner walls of a blood flow pathway 83 to hold the coil 2204 within the blood flow pathway 83. Additionally or alternatively, the sensor implant device 2200 may be anchored in a left atrial appendage and/or other area(s) of tissue. In some examples, the proximal portion 2209 of the coil 2204 may be shape-set to a greater diameter than other portions of the coil 2204. Additionally or alternatively, the proximal portion 2209 may be configured to be positioned at least partially outside of the blood flow pathway 83 to allow the proximal portion 2209 to expand beyond the diameter of the blood flow pathway 83. The proximal portion 2209 may be configured to prevent at least a portion of the sensor device 2202 from entering the blood flow pathway 83. Accordingly, the sensor device 2202 may advantageously be positioned outside the blood flow pathway 83 and/or within a heart chamber to obtain measurements related to blood flow within the chamber.

The sensor device 2202 may comprise at least one sensor component 2205. The sensor component(s) 2205 may comprise any type of sensor device as described in detail above. The sensor implant device 2200 may be configured to position the sensor component(s) 2205 at a target location within a body, which can include a heart chamber (e.g., a left atrium), an opening (e.g., a left atrial appendage) into and/or from a heart chamber, and/or a blood flow pathway (e.g., a coronary sinus). In some examples, the sensor implant device 2200 may be configured to position at least the sensor component(s) outside of the blood flow pathway 83.

In some examples, the sensor implant device 2200 may be configured for transseptal delivery from the right atrium into the left atrium 2. However, the sensor implant device 2200 may be configured for various delivery paths and/or for delivery to various blood pathways 83 and/or chambers within a heart.

The coil 2204 may be configured to position the sensor device 2202 at least partially beyond and/or outside of the blood flow pathway 83. For example, the coil 2204 may be configured to extend beyond the sensor device 2202 and/or may be configured to extend into the blood flow pathway 83 while the sensor device 2202 remains at least partially outside the blood flow pathway 83.

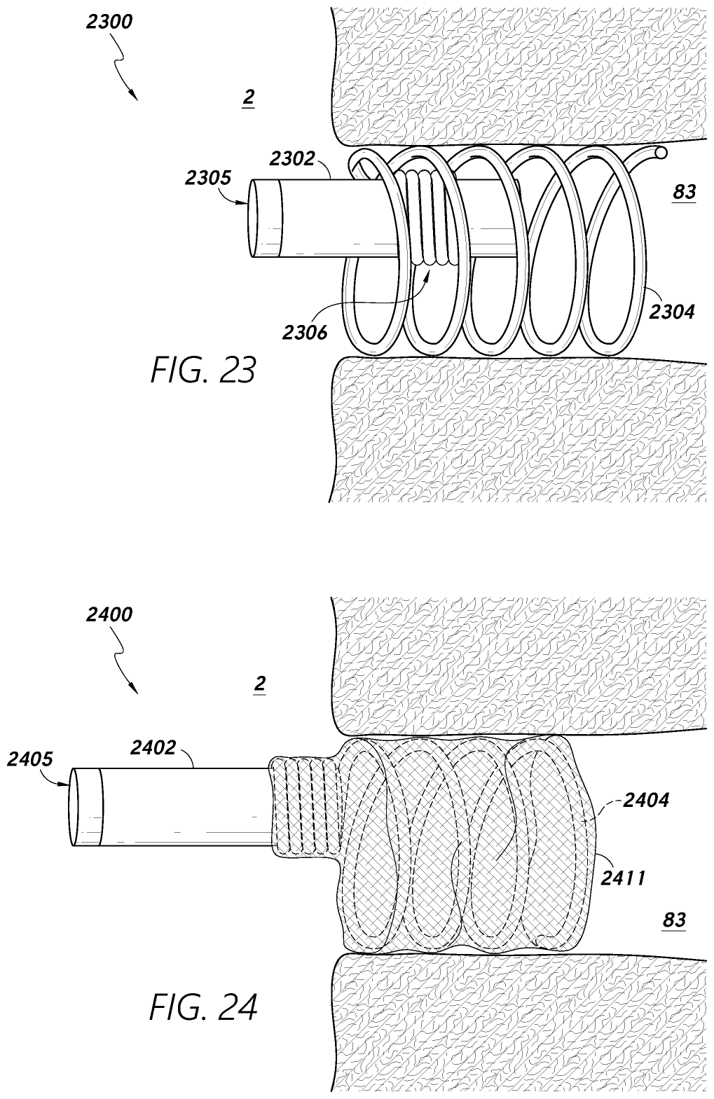

FIG. 23 illustrates a sensor implant device 2300 in accordance with one or more examples. The sensor implant device 2300 can comprise a sensor assembly/device 2302 coupled, attached, and/or otherwise releasably and/or permanently secured to a coil 2304 and/or anchoring feature(s). In some examples, the coil 2304 may comprise one or more winds configured to wrap at least partially around the sensor device 2302 and/or to pierce and/or otherwise anchor to a tissue wall. The coil 2304 may comprise a single wire forming multiple winds. The sensor device 2302 may be configured to fit at least partially and/or entirely within an inner lumen formed by the coil 2304. In some examples, the sensor device 2302 may be held in place by friction between the sensor device 2302 and the winds of the coil 2304. Additionally or alternatively, the sensor device 2302 may be coupled to and/or secured to the coil 2304. For example, the first end 2306 may be configured to form one or winds around the sensor device 2302 to securely couple to the sensor device 2302.

In some examples, the coil 2304 may have shape-memory properties and/or may be configured to naturally assume the form shown in FIG. 23 in response to removal of outside forces. The coil 2304 may be configured to be straightened and/or compressed to fit within a delivery device (e.g., a catheter). Upon removal from the catheter, the coil 2304 may be configured to form one or more generally circular winds. In some examples, the coil 2304 may be configured to expand in diameter until the coil 2304 presses against inner walls of a blood flow pathway 83 to hold the coil 2304 within the blood flow pathway 83. Additionally or alternatively, the sensor implant device 2300 may be anchored in a left atrial appendage and/or other area(s) of tissue.

The sensor device 2302 may comprise at least one sensor component 2305. The sensor component(s) 2305 may comprise any type of sensor device as described in detail above. The sensor implant device 2300 may be configured to position the sensor component(s) 2305 at a target location within a body, which can include a heart chamber (e.g., a left atrium 2), an opening (e.g., a left atrial appendage) into and/or from a heart chamber, and/or a blood flow pathway 83 (e.g., a coronary sinus). In some examples, the sensor implant device 2300 may be configured to position at least the sensor component(s) outside of the blood flow pathway 83.

In some examples, the sensor implant device 2300 may be configured for transseptal delivery from the right atrium into the left atrium 2. However, the sensor implant device 2300 may be configured for various delivery paths and/or for delivery to various blood pathways 83 and/or chambers within a heart.

The coil 2304 may be configured to position the sensor device 2302 at least partially beyond and/or outside of the blood flow pathway 83. For example, the coil 2304 may be configured to extend beyond the sensor device 2302 and/or may be configured to extend into the blood flow pathway 83 while the sensor device 2302 remains at least partially outside the blood flow pathway 83.

FIG. 24 illustrates a sensor implant device 2400 in accordance with one or more examples. The sensor implant device 2400 can comprise a sensor assembly/device 2402 coupled, attached, and/or otherwise releasably and/or permanently secured to a coil 2404 and/or anchoring feature(s). In some examples, the coil 2404 may comprise one or more winds configured to wrap at least partially around the sensor device 2402 and/or to pierce and/or otherwise anchor to a tissue wall. The coil 2404 may comprise a single wire forming multiple winds. The sensor device 2402 may be configured to fit at least partially and/or entirely within an inner lumen formed by the coil 2404 and/or to extend from a first end of the coil 2404. In some examples, the sensor device 2402 may be held in place by friction between the sensor device 2402 and the winds of the coil 2404. Additionally or alternatively, the sensor device 2402 may be coupled to and/or secured to the coil 2404. For example, the first end may be configured to form one or winds around the sensor device 2402 to securely couple to the sensor device 2402.

The coil 2404 may be at least partially enclosed by a covering 2411. The covering may be configured to enclose and/or extend across gaps between the windings of the coil 2404 and/or to prevent the coil 2404 from snagging on tissue. In some examples, the covering 2411 may be at least partially composed of fabric and/or other materials.

In some examples, the coil 2404 may have shape-memory properties and/or may be configured to naturally assume the form shown in FIG. 24 in response to removal of outside forces. The coil 2404 may be configured to be straightened and/or compressed to fit within a delivery device (e.g., a catheter). Upon removal from the catheter, the coil 2404 may be configured to form one or more generally circular winds. In some examples, the coil 2404 may be configured to expand in diameter until the coil 2404 presses against inner walls of a blood flow pathway 83 to hold the coil 2404 within the blood flow pathway 83. Additionally or alternatively, the sensor implant device 2400 may be anchored in a left atrial appendage and/or other area(s) of tissue.

The sensor device 2402 may comprise at least one sensor component 2405. The sensor component(s) 2405 may comprise any type of sensor device as described in detail above. The sensor implant device 2400 may be configured to position the sensor component(s) 2405 at a target location within a body, which can include a heart chamber (e.g., a left atrium 2), an opening (e.g., a left atrial appendage) into and/or from a heart chamber, and/or a blood flow pathway 83 (e.g., a coronary sinus). In some examples, the sensor implant device 2400 may be configured to position at least the sensor component(s) outside of the blood flow pathway 83.

In some examples, the sensor implant device 2400 may be configured for transseptal delivery from the right atrium into the left atrium 2. However, the sensor implant device 2400 may be configured for various delivery paths and/or for delivery to various blood pathways and/or chambers within a heart.

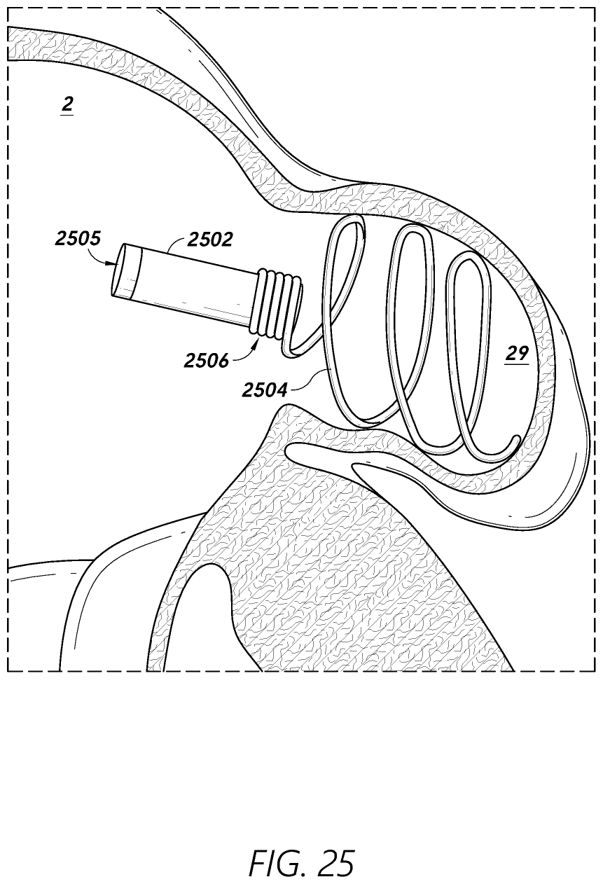

FIG. 25 illustrates a sensor implant device in accordance with one or more examples. The sensor implant device can comprise a sensor assembly/device 2502 coupled, attached, and/or otherwise releasably and/or permanently secured to a coil 2504 and/or anchoring feature(s). In some examples, the coil 2504 may comprise one or more winds configured to wrap at least partially around the sensor device 2502 and/or to pierce and/or otherwise anchor to a tissue wall. The coil 2504 may comprise a single wire forming multiple winds. The sensor device 2502 may be configured to fit at least partially within an inner lumen formed by the coil 2504 and/or to extend from a first end 2506 of the coil 2504 and/or at least partially out of and/or beyond the inner lumen of the coil 2504. In some examples, the sensor device 2502 may be held in place by friction between the sensor device 2502 and the winds of the coil 2504. Additionally or alternatively, the sensor device 2502 may be coupled to and/or secured to the coil 2504. For example, the first end 2506 may be configured to form one or winds around the sensor device 2502 to securely couple to the sensor device 2502.

In some examples, the coil 2504 may have shape-memory properties and/or may be configured to naturally assume the form shown in FIG. 25 in response to removal of outside forces. The coil 2504 may be configured to be straightened and/or compressed to fit within a delivery device (e.g., a catheter). Upon removal from the catheter, the coil 2504 may be configured to form one or more generally circular winds. In some examples, the coil 2504 may be configured to expand in diameter until the coil 2504 presses against inner walls of a left atrial appendage 29 to hold the coil 2504 within the left atrial appendage 29. Additionally or alternatively, the sensor implant device may be anchored in a left atrial appendage 29 and/or other area(s) of tissue.

The sensor device 2502 may comprise at least one sensor component 2505. The sensor component(s) 2505 may comprise any type of sensor device as described in detail above. The sensor implant device may be configured to position the sensor component(s) 2505 at a target location within a body, which can include a heart chamber (e.g., a left atrium 2), an opening (e.g., a left atrial appendage 29) into and/or from a heart chamber, and/or a blood flow pathway (e.g., a coronary sinus). In some examples, the sensor implant device may be configured to position at least the sensor component(s) outside of the left atrial appendage 29.

In some examples, the sensor implant device may be configured for transseptal delivery from the right atrium into the left atrium 2. However, the sensor implant device may be configured for various delivery paths and/or for delivery to various blood pathways and/or chambers within a heart.

The coil 2504 may be configured to position the sensor device 2502 at least partially beyond and/or outside of the left atrial appendage 29. For example, the coil 2504 may be configured to extend beyond the sensor device 2502 and/or may be configured to extend into the left atrial appendage 29 while the sensor device 2502 remains at least partially outside the left atrial appendage 29.

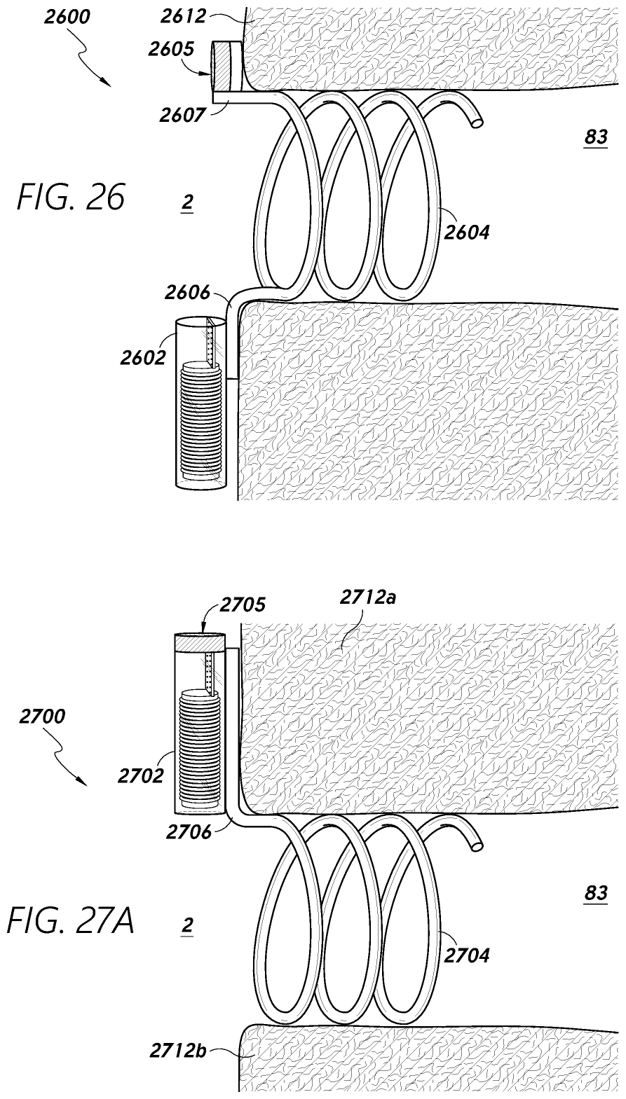

FIG. 26 illustrates a sensor implant device 2600 in accordance with one or more examples. The sensor implant device 2600 can comprise a sensor assembly/device including a sensor body 2602 and/or a sensor component 2605 coupled, attached, and/or otherwise releasably and/or permanently secured to a coil 2604 and/or anchoring feature(s). In some examples, the coil 2604 may comprise one or more winds configured to wrap at least partially around at least a portion of the sensor body 2602 and/or to pierce and/or otherwise anchor to a tissue wall. The coil 2604 may comprise a single wire forming multiple winds. The sensor device may be coupled to and/or secured to the coil 2604. For example, the sensor body 2602 may be coupled to a first end 2606 of the coil 2604 and/or the sensor component 2605 may be coupled to a second end 2607 of the coil 2604.

In some examples, the coil 2604 may have shape-memory properties and/or may be configured to naturally assume the form shown in FIG. 26 in response to removal of outside forces. The coil 2604 may be configured to be straightened and/or compressed to fit within a delivery device (e.g., a catheter). Upon removal from the catheter, the coil 2604 may be configured to form one or more generally circular winds. In some examples, the coil 2604 may be configured to expand in diameter until the coil 2604 presses against inner walls of a blood flow pathway 83 to hold the coil 2604 within the blood flow pathway 83. Additionally or alternatively, the sensor implant device 2600 may be anchored in a left atrial appendage and/or other area(s) of tissue.

The coil 2604 may be coupled to at least one sensor component 2605 that may be separate and/or distinct from the sensor body 2602. The sensor component(s) 2605 may comprise any type of sensor device as described in detail above. The sensor component 2605 and/or other sensor components may be configured to collect measurements related to blood flow. The sensor implant device 2600 may be configured to position the sensor component(s) 2605 and/or the sensor body 2602 at different target locations within a body, which can include a heart chamber (e.g., a left atrium), an opening (e.g., a left atrial appendage) into and/or from a heart chamber, and/or a blood flow pathway 83 (e.g., a coronary sinus). In some examples, the sensor implant device 2600 may be configured to position the sensor component(s) 2605 and/or the sensor body 2602 outside of the blood flow pathway 83. The coil 2604 may be configured to couple and/or interconnect the sensor component 2605 to the sensor body 2602. For example, the sensor component 2605 may be coupled and/or attached to a second end 2607 that may extend from and/or couple to the first end 2606. In some examples, the coil 2604 may be configured to position the sensor component 2605 at a first location (e.g., above an opening into the blood flow pathway 83 and/or adjacent a tissue wall 2612) and/or to position the sensor body 2602 at a second location (e.g., below the opening into the blood flow pathway 83 and/or adjacent a tissue wall 2612). One or more wires and/or the coil 2604 itself may be configured to form a transmission connection between the sensor component 2605 and the sensor body 2602.

The sensor body 2602 may comprise a housing configured to house one or more components for use in connection with the sensor component 2605. Example components housed at the sensor body 2602 can include an antenna (e.g., one or more coils or loops of conductive material, such as copper wire or the like), a transducer, control circuitry, and/or a battery.

In some examples, the sensor implant device 2600 may be configured for transseptal delivery from the right atrium into the left atrium 2. However, the sensor implant device 2600 may be configured for various delivery paths and/or for delivery to various blood pathways and/or chambers within a heart.

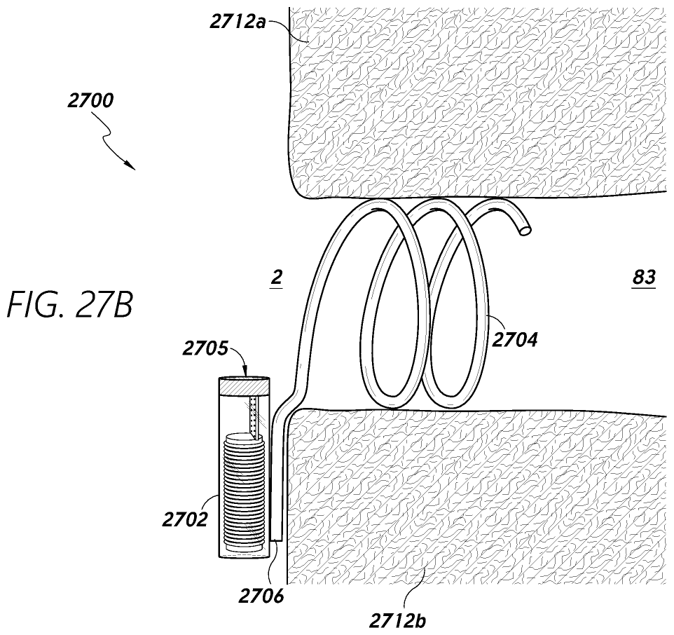

FIGS. 27A and 27B illustrate a sensor implant device 2700 in accordance with one or more examples. The sensor implant device 2700 can comprise a sensor assembly/device 2702 coupled, attached, and/or otherwise releasably and/or permanently secured to a coil 2704 and/or anchoring feature(s). In some examples, the coil 2704 may comprise one or more winds configured to wrap at least partially around the sensor device 2702 and/or to pierce and/or otherwise anchor to a tissue wall. The coil 2704 may comprise a single wire forming multiple winds. The sensor device 2702 may be configured to extend at least partially out of and/or beyond the inner lumen of the coil 2704 and/or blood flow pathway 83. In some examples, the sensor device 2702 may be configured to be positioned at least partially along and/or adjacent to a tissue wall 2712. The coil 2704 may be configured to be twisted to adjust which portion of the tissue wall 2712 the sensor device 2702 is adjacent to. For example, as shown in FIG. 27A, the sensor device 2702 may be situated adjacent to a first portion 2712a of the tissue wall. By twisting and/or otherwise adjusting the coil 2704, the sensor device 2702 may be moved adjacent to a second portion 2712b of the tissue wall, as shown in FIG. 27B. The sensor component 2705 may be situated near and/or extending at least partially over the blood flow pathway 83 (as shown in FIG. 27B) and/or may be situated distally from the blood flow pathway 83 (as shown in FIG. 27A).

In some examples, the sensor device 2702 may be coupled to and/or secured to the coil 2704. For example, a first end 2706 of the coil 2704 may be configured to extend along at least a portion of the sensor device 2702. In some examples, the first end 2706 may be situated between the sensor device 2702 and the tissue wall 2712.

In some examples, the coil 2704 may have shape-memory properties and/or may be configured to naturally assume the form shown in FIGS. 27A and 27B in response to removal of outside forces. The coil 2704 may be configured to be straightened and/or compressed to fit within a delivery device (e.g., a catheter). Upon removal from the catheter, the coil 2704 may be configured to form one or more generally circular winds. In some examples, the coil 2704 may be configured to expand in diameter until the coil 2704 presses against inner walls of a blood flow pathway 83 to hold the coil 2704 within the blood flow pathway 83. Additionally or alternatively, the sensor implant device 2700 may be anchored in a left atrial appendage and/or other area(s) of tissue.

The sensor device 2702 may comprise at least one sensor component 2705. The sensor component(s) 2705 may comprise any type of sensor device as described in detail above. The sensor implant device 2700 may be configured to position the sensor component(s) 2705 at a target location within a body, which can include a heart chamber (e.g., a left atrium 2), an opening (e.g., a left atrial appendage) into and/or from a heart chamber, and/or a blood flow pathway (e.g., a coronary sinus). In some examples, the sensor implant device 2700 may be configured to position at least the sensor component(s) outside of the blood flow pathway 83.

In some examples, the sensor implant device 2700 may be configured for transseptal delivery from the right atrium into the left atrium. However, the sensor implant device 2700 may be configured for various delivery paths and/or for delivery to various blood pathways and/or chambers within a heart.

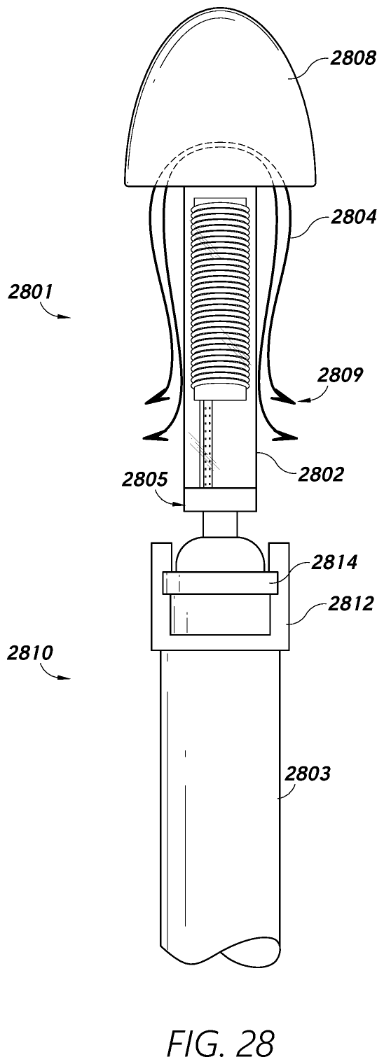

FIG. 28 illustrates a sensor implant device 2801 and at least a portion of a delivery system 2810 for delivering the sensor implant device 2801 and/or other devices in accordance with one or more examples. The sensor implant device 2801 can comprise a nose cone 2808 and/or similar feature that can be configured for providing a leading end of the sensor implant device 2801 and/or to lead and/or dilate one or more blood flow pathways to allow room for the sensor implant device 2801 and/or delivery systems 2810.

The sensor implant device 2801 may comprise a sensor device 2802, which can include any of the various sensor devices described herein. The sensor device 2802 may be coupled to and/or may comprise one or more sensor components 2805. The sensor device 2802 and/or sensor component(s) 2805 may be coupled to the nose cone 2808 and/or may extend at least partially into a hollow portion of the nose cone 2808.

The sensor implant device 2801 can further comprise one or more anchoring features 2804, which can include flexible arms having pointed tips 2809 and/or other features to enable the one or more anchoring features 2804 to penetrate and/or otherwise anchor to one or more areas of tissue. While the sensor implant device 2801 is shown comprising four anchoring features 2804, the sensor implant device 2801 can comprise any number of anchoring features 2804. In some examples, the one or more anchoring features 2804 may extend at least partially into a hollow interior of the nose cone 2808.

In some examples, one or more of the anchoring features 2804 may be at least partially composed of various shape-memory materials and/or alloys (e.g., Nitinol). For example, the one or more anchoring features 2804 may be shape-set to naturally extend to some degree away from the sensor device 2802 and/or from each other to enable the one or more anchoring features 2804 to contact and/or anchor to areas of tissue around the sensor implant device 2801 when the sensor implant device 2801 is delivered to a target location. Moreover, the one or more anchoring features 2804 may be configured to assume a compressed form (e.g., to press against and/or near the sensor device 2802) during delivery of the sensor implant device 2801. For example, the one or more anchoring features 2804 may be pressed inward and/or held in place by a catheter configured to extend over at least a portion of the sensor implant device 2801. In response to being removed from the catheter, the one or more anchoring features 2804 may be configured to extend outwardly and/or to engage the surrounding tissue.

The delivery system 2810 may be configured to removably couple to the sensor implant device 2801. In some examples, the delivery system 2810 may comprise a shaft 2803 coupled to and/or extending into a latch 2812 and/or other engagement feature. The latch 2812 may be configured to couple and/or otherwise secure to a corresponding notch 2814 and/or similar feature of the sensor implant device 2801. The notch 2814 may extend from and/or couple to the sensor component 2805 and/or sensor device 2802. In some examples, the nose cone 2808, sensor device 2802, sensor component 2805, notch 2814, and/or shaft 2803 may be coaxial. The latch 2812 may be configured to detach from the notch 2814 following removal of the sensor implant device 2801 from a catheter and/or other delivery systems.

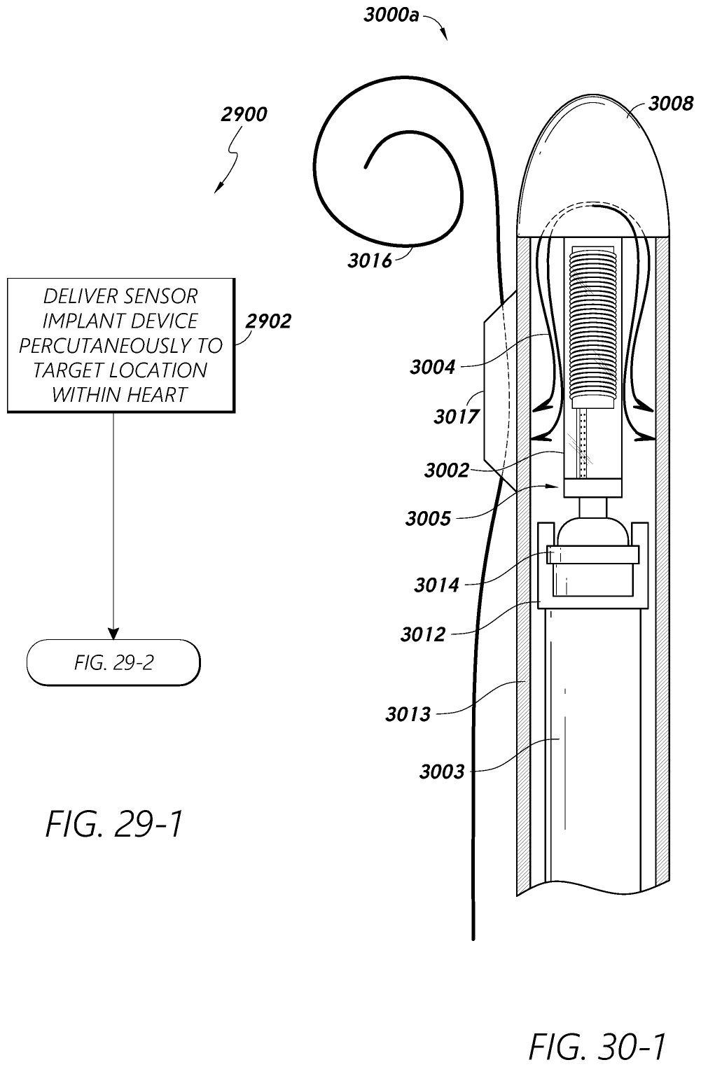

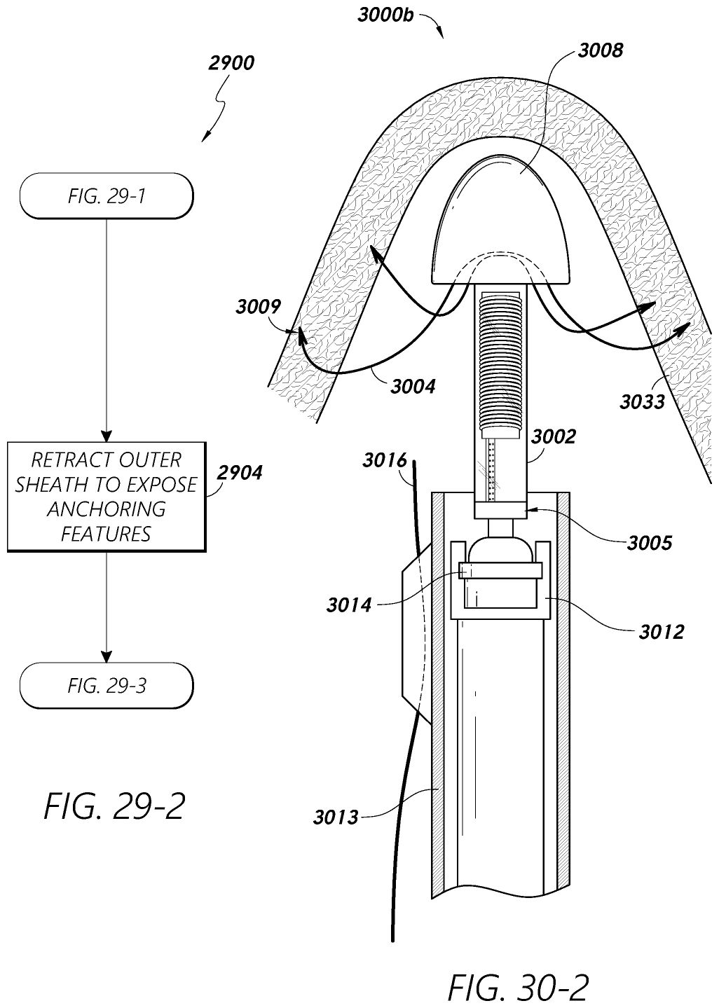

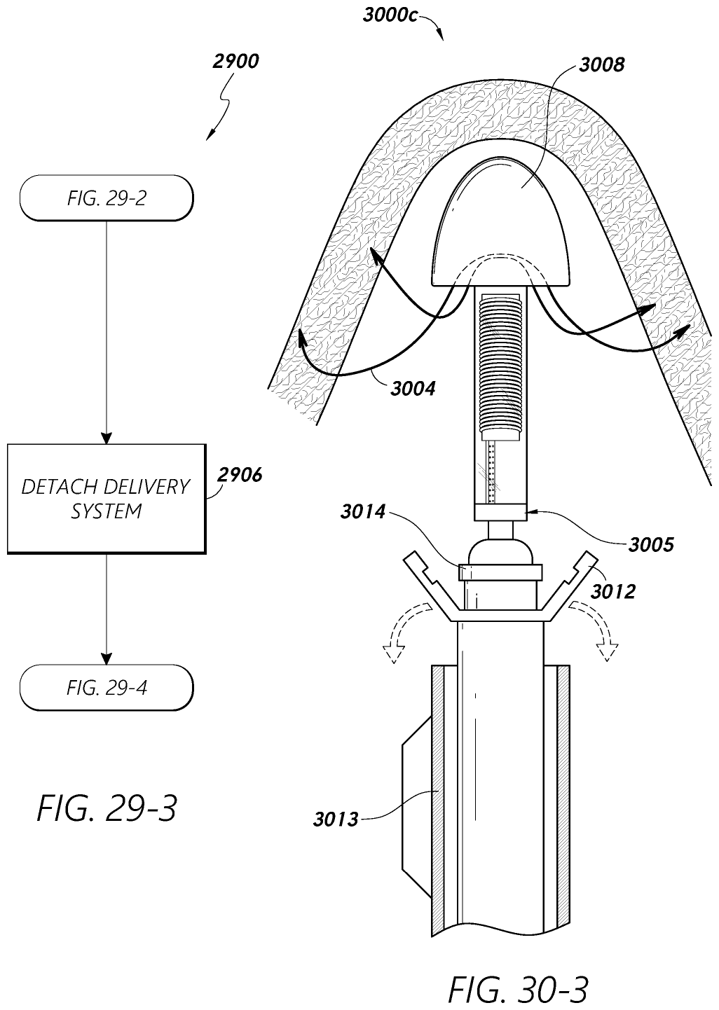

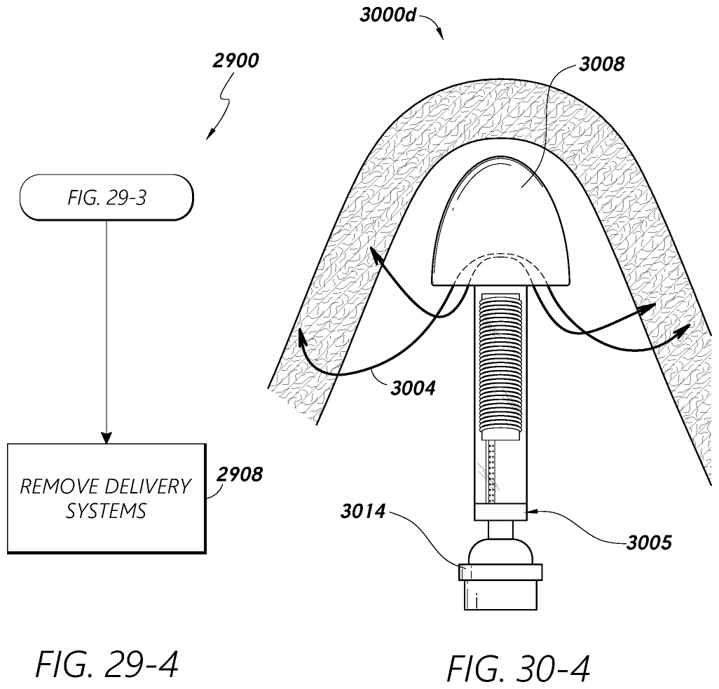

FIG. 29 (FIGS. 29-1, 29-2, 29-3, and 29-4) provides a flowchart illustrating a process 2900 including one or more steps for delivering one or more implants and/or sensors to target locations within a heart, in accordance with one or more examples. FIG. 30 (FIGS. 30-1, 30-2, 30-3, and 30-4) provides images corresponding to steps of the process 2900 of FIG. 29.

At step 2902, the process 2900 involves delivering a sensor implant device percutaneously to a target location within a heart via various delivery systems, which can include a catheter 3013, as shown in image 3000a of FIG. 30. The sensor implant device can comprise a nose cone 3008, one or more sensor devices 3002, one or more sensor components 3005, one or more anchoring features 3004, and/or a notch 3014 and/or similar mechanism for attaching to various delivery systems. The delivery systems can include a latch 3012 and/or a shaft 3003 configured to fit at least partially within the catheter 3013. The delivery systems may further comprise one or more guidewires 3016 configured to guide the catheter 3013 to a target location. In some examples, a guidewire receiver 3017 may extend from the catheter 3013 and/or may be configured to receive the guidewire 3016 to enable the catheter 3013 to attach to the guidewire 3016 and/or to slide along the guidewire 3016. The catheter 3013 may be configured to prevent the one or more anchoring features 3004 from expanding and/or extending away from the sensor device 3002.

At step 2904, the process 2900 involves at least partially retracting the catheter 3013 (i.e., “sheath” or “outer sheath”) to expose at least a portion of the sensor implant device including the one or more anchoring features 3004, as shown in image 3000b of FIG. 30. In some examples, the one or more anchoring features 3004 may be shape-set and/or may be configured to naturally extend away from the sensor device 3002 and/or towards one or more areas of tissue in response to retraction of the catheter 3013. The one or more anchoring features 3004 may comprise pointed tips 3009, which can include hooks, configured to penetrate and/or anchor to the surrounding tissue walls 3033. In some examples, the sensor implant device may be configured for delivery into the left atrial appendage and/or other portions of the anatomy. The sensor device 3002 may be configured to extend at least partially out of the left atrial appendage and/or other area of the heart that the one or more anchoring features may be anchored in.

At step 2906, the process 2900 involves detaching the delivery systems from the sensor implant device, as shown in image 3000c of FIG. 30. In some examples, the catheter 3013 may be further retracted to expose the latch 3012 and/or notch 3014 prior to detaching the delivery systems from the sensor implant device. The latch 3012 may be configured to enclose and/or latch onto at least a portion of the notch 3014 to attach the delivery systems to the sensor implant device. To detach the delivery systems, the latch 3012 may be pulled away from the notch 3014 to create a separation between the latch 3012 and the notch 3014. In some examples, pull wires and/or similar devices may be used to pull the latch 3012 away from the notch 3014.

At step 2908, the process 2900 involves removing the delivery systems while leaving the sensor implant device anchored at the target location, as illustrated in image 3000d of FIG. 30.

In accordance with some implementations of the present disclosure, a sensor implant device comprises a sensor body/device, a sensor component, and one or more anchoring features coupled to the sensor body and configured to anchor within a blood flow pathway or left atrial appendage.

The one or more anchoring features may comprise a first loop and a second loop extending from opposite sides of the sensor body. In some examples, the first loop comprises one or more barbs extending from the first loop and configured to anchor to a tissue wall.

In some examples, the first loop and the second loop are configured to naturally extend away from the sensor body. The first loop and the second loop may be configured to naturally extend towards each other.

The first loop may increase in diameter towards a distal portion of the first loop. In some examples, the first loop extends from a housing coupled to the sensor body.

In some examples, the sensor component is situated at a first end of the sensor body and the housing is situated at or near the first end of the sensor body. The sensor component may be situated at a first end of the sensor body and the housing may be situated at or near a second end of the sensor body distal from the first end.

The first loop and the second loop may be configured to extend from a first end of the sensor body and may be configured to extend along a length of the sensor body towards a second end of the sensor body. In some examples, the first loop and the second loop are configured to extend from a first end of the sensor body and are configured to extend generally in parallel with the sensor body and away from a second end of the sensor body.

In some examples, the first loop is configured to anchor into a first blood flow pathway and the second loop is configured to anchor into a second blood flow pathway. The sensor component may be configured to be situated outside the first blood flow pathway and the second blood flow pathway.

The first loop may be configured to anchor into a first blood flow pathway and the second loop may be configured to anchor into the first blood flow pathway. In some examples, the sensor component is configured to be situated outside the first blood flow pathway.

In some examples, the one or more anchoring features comprise a stent having an inner lumen, and wherein the sensor body is situated at least partially within the inner lumen. Most of the sensor body may extend beyond the inner lumen of the stent.

Most of the sensor body may be situated within the inner lumen of the stent. In some examples, the sensor component is configured to extend beyond the inner lumen of the stent.

In some examples, the one or more anchoring features comprise a C-shaped clip configured to wrap at least partially around the sensor body. The clip may comprise two pointed ends configured to anchor to a tissue wall.

The one or more anchoring features may comprise a coil configured to at least partially encircle the sensor body and configured to penetrate multiple portions of a tissue wall. In some examples, the sensor component is configured to extend beyond an inner lumen formed by the coil.

In some examples, the one or more anchoring features comprise two or more hooks configured to extend approximately in-line with a length of the sensor body while within a delivery device. The one or more anchoring features may be configured to naturally extend away from the sensor body in response to removal from the delivery device.

The one or more anchoring features may comprise a coil configured to hold the sensor body at least partially beyond the blood flow pathway or left atrial appendage. In some examples, the coil comprises a first portion configured to anchor within the blood flow pathway or left atrial appendage by expanding to press against the blood flow pathway or left atrial appendage.

In some examples, the coil comprises a second portion having a greater width than the first portion and configured for placement beyond the blood flow pathway or left atrial appendage. The sensor implant device may further comprise a covering enclosing the coil.

The sensor body may be separate from the sensor component. In some examples, the sensor implant device further comprises a nose cone configured to lead the sensor body during delivery to the blood flow pathway or left atrial appendage.

In some examples, the one or more anchoring features comprise two or more arms having pointed tips and configured to extend away from the sensor body. The sensor implant device may further comprise a notch coupled to the sensor body and configured to receive a latch of a delivery system.

Some implementations of the present disclosure relate to a method comprising delivering a sensor implant device in a compressed form to a blood flow pathway or left atrial appendage via a delivery device. The sensor implant device comprises a sensor body, a sensor component, and one or more anchoring features coupled to the sensor body. The method further comprises removing the sensor implant device from the delivery device to cause the sensor implant device to naturally assume an expanded form and anchoring the one or more anchoring features to the blood flow pathway or left atrial appendage.

In some examples, the sensor implant device further comprises a notch. The method may further comprise securing a latch of a delivery system to the notch and detaching the latch from the notch following removal of the sensor implant device form the delivery device.

In some examples, the one or more anchoring features comprise a first loop and a second loop extending from opposite sides of the sensor body. The first loop may comprise one or more barbs extending from the first loop and configured to anchor to a tissue wall.

The first loop and the second loop may be configured to naturally extend away from the sensor device. In some examples, the first loop and the second loop are configured to naturally extend towards each other.

In some examples, the one or more anchoring features comprise a stent having an inner lumen. The sensor body may be situated at least partially within the inner lumen.

The one or more anchoring features may comprise a C-shaped clip configured to wrap at least partially around the sensor body. In some examples, the one or more anchoring features comprise a coil configured to at least partially encircle the sensor body and configured to penetrate multiple portions of a tissue wall.

In some examples, the one or more anchoring features comprise two or more hooks configured to extend approximately in-line with a length of the sensor body while within a delivery device. The one or more anchoring features may be configured to naturally extend away from the sensor body in response to removal from the delivery device.

The one or more anchoring features may comprise a coil configured to hold the sensor body at least partially beyond the blood flow pathway or left atrial appendage. In some examples, the coil comprises a first portion configured to anchor within the blood flow pathway or left atrial appendage by expanding to press against the blood flow pathway or left atrial appendage.

In some examples, the coil comprises a second portion having a greater width than the first portion and configured for placement beyond the blood flow pathway or left atrial appendage. The method may further comprise a covering enclosing the coil.

The sensor body may be separate from the sensor component. In some examples, the method further comprises a nose cone configured to lead the sensor body during delivery to the blood flow pathway or left atrial appendage. The one or more anchoring features may comprise two or more arms having pointed tips and configured to extend away from the sensor body.

### ADDITIONAL EXAMPLES

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A sensor implant device comprising:
a sensor body;
a sensor component; and
one or more anchoring features coupled to the sensor body and configured to anchor within a blood flow pathway or left atrial appendage.

2. The sensor implant device of claim 1, wherein the one or more anchoring features comprise a first loop and a second loop extending from opposite sides of the sensor body.

3. The sensor implant device of claim 2, wherein the first loop extends from a housing coupled to the sensor body.

4. The sensor implant device of claim 2, wherein the first loop is configured to anchor into a first blood flow pathway and the second loop is configured to anchor into a second blood flow pathway.

5. The sensor implant device of claim 2, wherein the first loop is configured to anchor into a first blood flow pathway and the second loop is configured to anchor into the first blood flow pathway.

6. The sensor implant device of claim 1, wherein the one or more anchoring features comprise a stent having an inner lumen, and wherein the sensor body is situated at least partially within the inner lumen.

7. The sensor implant device of claim 1, wherein the one or more anchoring features comprise a C-shaped clip configured to wrap at least partially around the sensor body.

8. The sensor implant device of claim 1, wherein the one or more anchoring features comprise a coil configured to at least partially encircle the sensor body and configured to penetrate multiple portions of a tissue wall.

9. The sensor implant device of claim 1, wherein the one or more anchoring features comprise two or more hooks configured to extend approximately in-line with a length of the sensor body while within a delivery device.

10. The sensor implant device of claim 1, wherein the one or more anchoring features comprise a coil configured to hold the sensor body at least partially beyond the blood flow pathway or left atrial appendage.

11. The sensor implant device of claim 1, further comprising a nose cone configured to lead the sensor body during delivery to the blood flow pathway or left atrial appendage.

12. A method comprising:
delivering a sensor implant device in a compressed form to a blood flow pathway or left atrial appendage via a delivery device, the sensor implant device comprising:
a sensor body;
a sensor component; and
one or more anchoring features coupled to the sensor body;
removing the sensor implant device from the delivery device to cause the sensor implant device to naturally assume an expanded form; and
anchoring the one or more anchoring features to the blood flow pathway or left atrial appendage.

13. The method of claim 12, wherein the sensor implant device further comprises a notch, and wherein the method further comprises securing a latch of a delivery system to the notch, and detaching the latch from the notch following removal of the sensor implant device form the delivery device.

14. The method of claim 12, wherein the one or more anchoring features comprise a first loop and a second loop extending from opposite sides of the sensor body.

15. The method of claim 12, wherein the one or more anchoring features comprise two or more hooks configured to extend approximately in-line with a length of the sensor body while within a delivery device.

16. The method of claim 12, wherein the one or more anchoring features comprise a coil configured to hold the sensor body at least partially beyond the blood flow pathway or left atrial appendage.

17. The method of claim 16, wherein the coil comprises a first portion configured to anchor within the blood flow pathway or left atrial appendage by expanding to press against the blood flow pathway or left atrial appendage.

18. The method of claim 12, wherein the sensor body is separate from the sensor component.

19. The method of claim 12, further comprising a nose cone configured to lead the sensor body during delivery to the blood flow pathway or left atrial appendage.

20. The method of claim 19, wherein the one or more anchoring features comprise two or more arms having pointed tips and configured to extend away from the sensor body.
